(19) United States
(12) Patent Application Publication
(10) Pub. No.: US 2020/0360563 A1
(43) Pub. Date: Nov. 19, 2020

(54) Methods for Vascular Construction and Products Therefrom

(71) Applicant: University of Georgia Research Foundation, Inc., Athens, GA (US)

(72) Inventors: Stephen Dalton, Athens, GA (US); Miranda Kristine Hayworth, Galveston, TX (US)

(21) Appl. No.: 16/956,362

(22) PCT Filed: Jan. 30, 2019
(86) PCT No.: PCT/US2019/015792

§ 371 Date: Jun. 19, 2020

Related U.S. Application Data
(60) Provisional application No. 62/623,760, filed on Jan. 30, 2018.

(51) Int. Cl. A61L 27/36; C12N 5/071; A61K 35/44; A61L 27/24; A61L 27/38; A61L 27/50

(52) U.S. Cl. CPC A61L 27/3625; C12N 5/0691; A61K 35/44; A61L 27/24; A61L 27/3808; A61L 2300/414; A61L 27/507; C12N 2533/54; C12N 2501/105; C12N 2501/115; A61L 27/3826

(57) ABSTRACT

The present invention is directed to vascular tissue constructs or vessels and methods for producing vascular tissue constructs or vessels (ie., fabricated blood vessels), including veins, arteries, capillaries and other vascular structures from a biomaterial foundation or scaffold and epicardial progenitor cells (EPCs) which are seeded onto the biomaterial, exposed to a differentiation medium and differentiated into endothelial, cells, smooth muscle cells and pericytes which self-assemble into vascular tissue associated with N the biomaterial foundation or scaffold.

### RELATED APPLICATIONS

This application claims the benefit of priority of U.S. provisional application No. US62/623,750 of identical title, filed Jan. 30, 2018, the entire contents of which application is incorporated by reference herein.

This invention was made with government support under grants P01 HL089471 and P01 GM75334 awarded by the National Institutes of Health (NIH). The government has certain rights in the invention.

### FIELD OF THE INVENTION

The present invention is directed to vascular tissue constructs or vessels and methods for producing vascular tissue constructs or vessels (ie., fabricated blood vessels), including veins, arteries, capillaries and other vascular structures from a biomaterial foundation or scaffold) (often the material is a decellularized tubular tissue such as a blood vessel, other vascular material or material from an intestine obtained from a mammalian source, or an electrospun or 3D printed biomaterial) and epicardial progenitor cells (MesoTs/EPCs) which are seeded onto the biomaterial, exposed to a differentiation medium and differentiated into endothelial cells, smooth muscle cells and pericytes associated with the biomaterial foundation or scaffold. It has been discovered that epicardial progenitor cells, which are differentiated in biomaterial scaffolds according to the present invention self-assemble into vascular networks to provide biological vascular structures which resemble natural blood vessels. These vascular constructs of the present invention can be used in place of natural blood vessels for a number of biomedical applications. The vascular structures pursuant to the present invention find use in surgery (to repair and/or replace diseased, damaged and/or impaired blood vessels or other vascular tissue), for disease modeling (principally a variety of vascular and other diseases) in drug discovery/development, and laboratory testing applications.

### BACKGROUND AND OVERVIEW OF THE INVENTION

Coelomic organs including the heart, spleen, lungs, liver and gut are lined on their outer surface by a thin layer of cells with epithelia characteristics known as visceral mesothelium (Mutsaers and Wilkosz, 2007). During early development, mesothelium is highly dynamic and critical for growth and maintenance of the underlying tissue. Following the formation of the mesothelial layer, a subpopulation of these cells undergoes an epithelial to mesenchymal transition (EMT) and invade the underlying tissue. Here, they transition through a mesenchymal progenitor intermediate and in response to local signals they differentiate into vascular lineages, which contribute to a nascent vascular network (Asahina et al., 2009; Cano et al., 2013; Dixit et al., 2013; Que et al., 2008; Rinkevich et al., 2012; Smith et al., 2011; Wilm et al., 2005; Zangi et al., 2013). Mesothelium-derived progenitor cells with mesenchymal characteristics have been described in the heart (Chong et al., 2011; Rinkevich et al., 2012; Zangi et al., 2013), gut, lungs and liver (Rinkevich et al., 2012) and contribute to vascularization of these organs during embryonic development and possibly during tissue regeneration (Kikuchi et al., 2011; Smart et al., 2011). Numerous reports have also highlighted the broad potential of mesothelium and mesothelium-derived cells in neo-vascularization and tissue regeneration (Elmadbouh et al., 2005; Lucas, 2007; Mutsaers and Wilkosz, 2007; Shelton and Bader, 2012). For example, the epicardium and its derivatives have been implicated in regenerative responses following myocardial infarction (Elmadbouh et al., 2005; Smart et al., 2011; van Wijk et al., 2012) and mechanical injury (Porrello et al., 2011).

Immediately following ischemic events, re-establishment of blood flow is crucial to prevent further tissue damage and organ functional decline. Endogenous repair mechanisms rely on the resident vascular progenitor population, including mesothelium, to achieve this. Recently, considerable progress has been made to generate vascular lineages (Cheung et al., 2012; James et al., 2010; Liu et al., 2014; Patsch et al., 2015) in vitro. While smooth muscle cells (SMCs)(Cheung et al., 2012; Liu et al., 2014; Patsch et al., 2015) and endothelial cells (ECs)(James et al., 2010; Patsch et al., 2015) have been directly generated from human pluripotent stem cell (hPSC)-derived mesoderm, a multipotent progenitor equivalent to a tissue resident vascular precursor has not been reported. The availability of such a progenitor cell could have utility in revascularization of damaged tissue or potentially in vascular engineering (Colunga and Dalton, 2018). Thus, we attempted to recapitulate the developmental program of mesothelium in order to identify such a cell type in vitro.

This application relates to the generation of a multipotent, vascular progenitor cell of the mesothelium lineage (which are labelled “MesoT” cells) from hPSCs, which can be further differentiated. MesoT cells exhibit the ability to generate all the required vascular lineages such as SMCs. ECs, and pericytes which is consistent with reported tissue resident progenitor cells of the coelomic organs. The present invention also relates to the ability of MesoT cells to revascularize damaged tissue and exhibit its potential utility in tissue engineering contexts. This is of significance because current tissue engineering approaches for generating transplantable blood vessels have been largely limited by the availability and reliability of vascular cells with proven utility. The availability of hPSC-derived, multipotent vascular progenitors of the mesothelium lineage opens up new opportunities for the advancement of tissue engineering and regenerative medicine.

Generation of synthetic vessels for vascular reconstruction has been partially successful using large diameter (˜>5 mm) fabricated vessels. However, no effective method of generating medium to small vessels (˜<5 mm) has been described in the art. This is because fabricated vessels fail within a few months of transplantation due to loss of function, clotting and inflammation. Vascular engineering for most medical situations is therefore quite limited. The vessels to which the present application is directed and will generate are suitable to fill a large gap in biomedical engineering relating to vascular disease and injury. Applications of the present invention include the treatment of diabetic patients with peripheral vascular disease, patients with atherosclerotic occlusions, aneurysms and in tissue reconstruction following traumatic injury or surgery.

Previous attempts to generate functional vessels for transplantation use combinations of mature endothelial cells, smooth muscle cells, mesenchymal stem cells and fibroblasts. When these cells are seeded into a variety of scaffolds the resulting constructs generally fail and are not suitable for clinical use. The use of MesoTs/EPCs as a cell source pursuant to the present invention solves this massive biomedical problem.

The present invention is a combination of stem/progenitor technology (epicardium progenitor cell, EPC) developed by the Dalton laboratory at UGA combined with biomaterial scaffolds for generation of transplantable vasculature. In vivo transplantation experiments using decellularized vascular constructs have been performed and the strategy shows excellent efficacy. Electrospun scaffolds and 3D printed biomaterials are also useful for similar purposes. Thus, Applicant shows that a defined, decellularized vascular construct modified pursuant to the present invention can function in vivo—for example, a decellularized pig vessel of <5 mm in diameter that has been transplanted into an animal model for several weeks. We also show utility for an electrospun biomaterials construct seeded with MesoTs/EPCs that function in an animal model.

### SUMMARY OF THE INVENTION

The present invention is directed to a method of producing vascular tissue constructs or vessels from scaffolds (e.g. decellularized tubular, especially vascular tissue, electrospun polymer scaffolds or 3D printed polymer scaffolds) which are seeded with human pluripotent stem cell-derived vascular progenitor cell of the mesothelium lineage (migratory mesoderm or MesoTs), exposed to a vascular differentiation medium to differentiate the MesoTs into a endothelial cells, smooth muscle cells and pericytes which self-assemble into vascular networks to produce functional blood vessels. In the present invention, human pluripotent stem cell-derived vascular progenitor cell of the mesothelium lineage MesoT cells or epicardial progenitor cells (hereinafter, these cells will be referred to as MesoTs, EPCs or MesoTs/EPCs) as described herein are seeded onto the biomaterials scaffold, exposed to a vascular differentiation medium which contains nutrients and growth factors which facilitate the growth of these MesoT pluripotent cells into vascular lineages (at least endothelial and smooth muscle cells and preferably, endothelial cells, smooth muscle cells and pericytes) and the MesoTs are seeded onto the surface of the biomaterial (often through the pedicles of the scaffold into the vascular tree) over a period of several hours to S days (generally about 2-5 days or about 2-4 days, often 3 days) to fuse the cells onto the surface of the scaffold, and differentiated over a course of from 1 to 12 weeks or more, often 1 to 6 weeks (often 14-30 days) in association with the scaffold in the vascular differentiation medium to produce vascular lineages comprising a population of endothelial cells and smooth muscle cells and optionally pericytes in combination with endothelial cells and smooth muscle cells in association with the scaffold which have unexpectedly self-assembled into a cellularized vascular construct which functions as a natural blood vessel. Thus, in the present invention MesoTs which are derived from human pluripotent stem cells (hPSCs) are used in conjunction with an initial scaffold which is free from cellular material (e.g., decellularized biological scaffolds such as tubular tissue, including vascular tissue from a human, non-human primate, pig or cow, among others, electrospun polymer scaffolds or 3D printed polymer scaffolds) to generate cellularized constructs which comprise the initial scaffold and a combination of endothelial cells and smooth muscle cells and optionally pericytes in association with the scaffold which are self-assembled into a functional blood vessel.

The vessels which are provided by the present invention are useful for vascular engineering of organs and tissue including heart/cardiovascular tissue, kidney, brain and central nervous system tissue. In addition, the vessels which are produced according to the present invention are useful in the repair and/or replacement of vessels following vascular occlusion, diseased blood vessels (coronary infarction, diabetes—heart, renal, brain, central nervous system, pancreatic and other tissues, atherosclerosis, traumatic injury, aneurysms and surgery, especially including heart and vascular surgery).

In an embodiment, the present invention is directed to a kit which comprises tubular biomaterial, a population of MesoTs and differentiation medium with instructions for seeding the tubular biomaterial with the MesoTs and differentiating the MesoTs with the differentiation medium into endothelial cells, smooth muscle cells and pericytes which self-assemble into vascular tissue in association with the tubular biomaterial forming a vessel.

In an embodiment, the present invention is directed to a method for revascularizing damaged blood vessels and other vascular tissue by seeding the damaged blood vessels and/or other vascular tissue with MesoT cells in combination (e.g. by suspending cells in differentiation media prior to seeding) with a differentiation medium (preferably chemically defined media or CDM minus Activin A) in combination with an effective amount of a VEGF agonist (e.g. a VEGF pathway agonist or a VEGF receptor agonist), preferably VEGF-A₁₆₅ for a period of at least 8-10 days, preferably at least 12 days to produce endothelial cells, smooth muscle cells and often pericytes which will self-assemble into vascular tissue, thus repairing the damaged blood vessels and/or vascular tissue.

### BRIEF DESCRIPTION OF THE FIGURES

FIG. 1 (A) shows migratory mesodermal cells/epicardial progenitor cells (MesoTs/EPCs) differentiate into vascular lineages and self-assemble into vessel like networks in vitro when cultured in minimum essential media containing growth factors and other components. In this figure the MesoTs/EPCs were differentiated in DMEM/F12 media supplemented with heregulin β, IGF, FGF2 and VEGF over 12-14 days. The differentiation was evidenced by SMA, smooth muscle actin; vWF, von Willebrand factor; DAPI (DNA). (B) The results set forth in this figure evidence that EPC vascular progenitors can differentiate into endothelial cells, smooth muscle cells and pericytes and self-assemble into vascular networks in vitro.

FIG. 2 shows the system used to evaluate vascular activity of MesoTs/EPCs in a decellularized vessel system. (A) Decellularized rat jejenum BioVac before perfusion with cells. (B) BioVac system enclosed within a temperature and gas-controlled incubator.

FIG. 3. Decellularized BioVac construct obtained was seeded with MesoTs/EPCs for 28 days and stained with MTT dye that recognizes viable cells (purple). The figure indicates that viable cells have lined the vasculature network. (Top) at low magnification; (Bottom) higher magnification of image above (as indicated by box).

FIG. 4 shows the light sheet microscopy of recellularized vascular tree subject to IF analysis by probing with (a) CD31 antibody (scale bar 20 microns), (b) CD31 and SMA (scale bar, 25 microns), (c) NG2 (scale bar, 150 microns) and (d) NG2 and SMA (scale bar, 25 microns).

FIG. 5 shows the retention of FITC-dextran in vessels formed following EPC perfusion of BioVacs. Left panel; bright field view of the vessel. Right hand panel: FITC-dextran retention. Scale bar, 100 microns. Left, bright field. Right, immunofluorescence image of FITC-dextran.

FIG. 6 shows seeded BioVac-LDL uptake into endothelial cells (red) in vessels generated by seeding decellularized vessels with MesoTs/EPCs.

FIGS. 7 AND 8 show the functional integrity of the construct vasculature following transplantation for 3 days. The sites of anastomosis, blood-filled large vessels and capillaries of the graft vasculature are indicated.

FIG. 9 shows sections of EPC-seeded graft that recovered 3 days after transplantation. Sections were probed with antibodies recognizing NG2, SMA and CD31 as indicated.

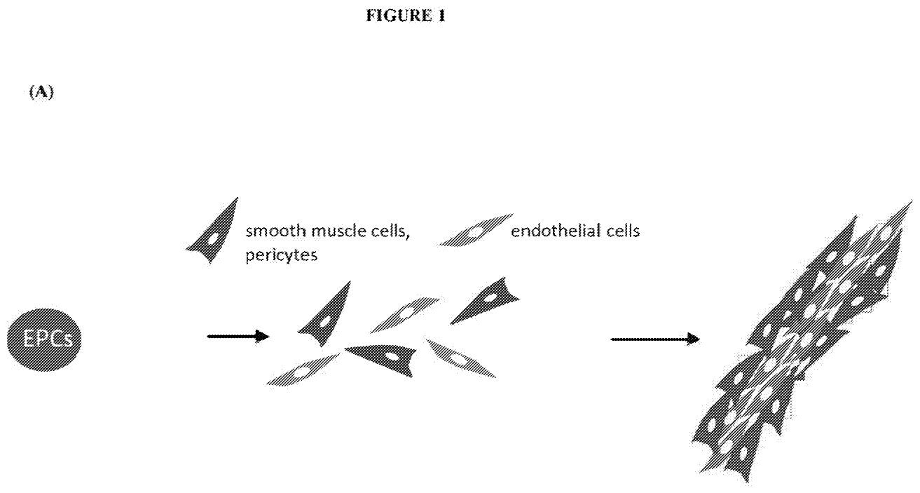

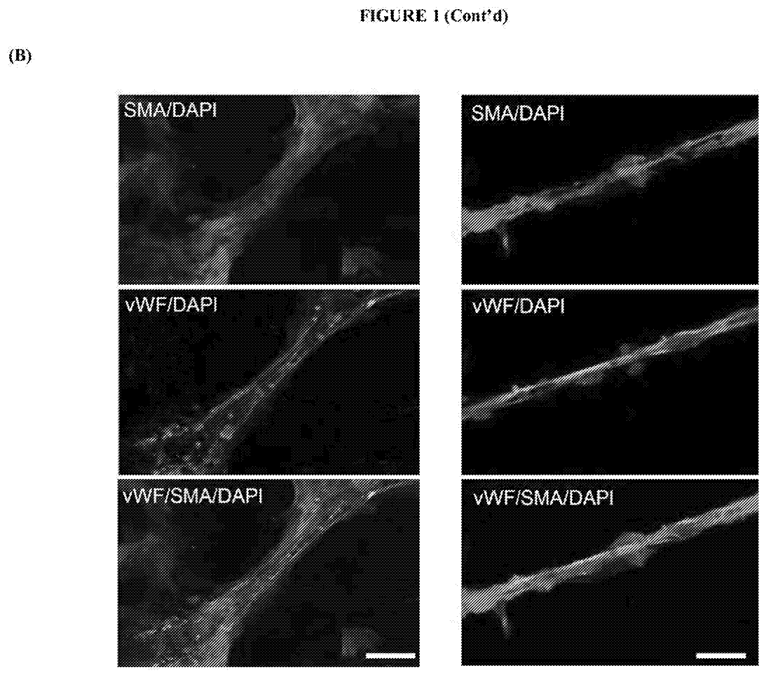

FIG. M1 shows hPSC-derived MesoT displays molecular characteristics of primary mesothelium. (A) Sources of cells used for RNA-seq analysis. hPSC-derived MesoT cells and tdTomato+mesothelium isolated from mouse embryonic gut, liver, heart and lungs (E15.5) were compared to RNA-seq data in the public domain. (B) Addition of Wnt3a, BMP4, and retinoic acid (RA) to SplM (ISL1⁺, NKX2.5⁺, ZO1⁺) efficiently generates MesoT (WT1⁺, αSMA⁺, VIM⁺) with a mesenchymal phenotype. Scale bars, 50 μm. (C) qRT-PCR data showing fold-change of transcript levels for markers of SplM (SL, NKX2.5 and GATA4) and MesoT (WT1, TBX18 and TCF21) following directed differentiation of human embryonic stem cells (hESCs, WA09). TaqMan assays for each transcript were performed in technical triplicate and fold-change shown relative to untreated hESCs (WA09) after normalization with 18S RNA. (D) Left: hierarchical clustering (Euclidean distance, complete linkage) of all human (black box) and mouse (red box) RNA-seq samples according to mouse and human orthologs. Data from both species were log transformed and scaled to mean=0 and standard deviation=1 prior to clustering. Right: zoomed-in section of the highlighted portion of the array tree dendrogram (left panel) showing that hPSC-derived MesoT are closely related to human (h) and adult (a) epicardium; mouse (m) and fetal (f) mesothelium. Replicate numbers from independent experiments are indicated. Error bars±standard deviation. See also FIGS. S1, S2 and Table S4.

FIG. M2 shows that epigenetic and transcript profiles of MesoT are similar to vascular cell types. (A) Hierarchical clustering (Euclidean distance, complete linkage) of human tissue and hESC-derived samples according to Beta values for the 1846 cytosines comprising module 9 of the DNA methylation profile. Array tree dendrograms and the distribution of Beta values for these cytosines are presented in heatmap form (top) and as box and whisker plots (bottom). (B) Illustration depicting the epigenetic landscape at primed and activated enhancers as MesoT cells transition to a vascular fate. Top portion depicts vascular genes ‘primed’ in MesoT with the presence of K4me1 on histone H3 at enhancer sites. Bottom portion depicts the primed enhancers for vascular genes being activated by addition of K27ac as they differentiate to smooth muscle cells (SMCs) or endothelial cells (EC). (C) Enhancer and gene ontology discovery pipeline for data in (D; FIGS. S3B-S3D). (D) Heatmaps of H3K4me1 (red) and H3K27ac (blue) ChIP-seq data for enhancers linked to up-regulated genes in primary smooth muscle and endothelial cells, compared to MesoTs. (E) Principal component analysis of the top 50% of highly expressed genes in hESC-derived mesothelium cells (MesoT and MLC), primary fetal tissue (liver, heart, brain, spinal cord and hindbrain) and primary cells (Endo and SMC). MesoT and MesoT (FCS)(self-renewing) cells cluster tightly with vascular cell types (Endo and SMC) and hESC-derived mesothelium (MLC). See also FIG. S3 and Tables S1 and S4.

FIG. M3 shows MesoT efficiently differentiate to smooth muscle and endothelial cells. (A) MesoT cells treated with PDGF-BB (50 ng/ml) in CDM-Activin A for 12 days were probed with antibodies for alpha smooth muscle actin (αSMA), calponin, and myosin heavy chain 11 (MYH11). Scale bars, 100 μm (left) and 50 μm (right). (B) MesoTs grown in CDM (-A) supplemented with PDGF-BB or VEGF-A₁₆₅ and SB431542 were quantified based on expression of lineage specific markers for SMCs and ECs. (C) DiO labeled SMCs as shown in (A) were treated with 100 μM carbachol or 50 mM KCl to stimulate functional contraction. (D) SMC surface area was measured after treatment (C). Contraction is shown as the % change in cell surface area for individual cells. Each treatment group was compared to corresponding control time point to determine statistical significance. N=20. (E-F) MesoT cells treated with CDM (-A), VEGF-A₁₆₅ and SB431542 for 12 days were fixed and probed with antibodies for CD31, vWF, and DAPI or, (F) characterized by flow cytometry with VE-cadherin (blue) or isotype control (red). Scale bar, 50 μm. (G) Trans-endothelial electrical resistance (TEER) was measured after culturing MesoT cells in CDM (-A) supplemented with VEGF-A₁₆₅ alone (+VEGF) or with SB431542(+VEGF+SB) after 14, 21 and 28 days and compared against primary dermal microvascular endothelium (1° ECs). N=3. (H) Barrier integrity was tested by measuring FITC-dextran (40 kDa) perfusion from the apical to basolateral side. Control represents the absence of cells. No statistical significance was determined when comparing cells to 1° ECs. (1) Schematic of the bioreactor culture system used in (G) and (H). (J) Immunofluorescence of cell monolayer as in (I) showing expression of tight junction marker ZO1 (top) and H&E staining (bottom). (K) Transmission electron microscopy image of MesoT-derived endothelium. Red arrows depict tight cell junctions. Inset (top) is depicted on bottom. Scale bars, 500 nm and 200 nm, respectively. ** p-value=0.0027 for two-way ANOVA. **** p-value<0.0001 for one-tailed t-test. Error bars±SEM. See also FIG. S4.

FIG. M4 shows that MesoTs are multipotent vascular progenitor cells. (A) Population doubling of MesoT (FBS) cells. Time=0 is when cells are first passed into serum containing media. Simple linear regression analysis (blue dashes) applied to data shows a tightly fitted regression line with a coefficient of determination (R²)=0.9774 and slope of 0.8282+/−0.0563. Experiment N=3 performed in technical triplicate. (B) Cell cycle analysis of self-renewing MesoT (FBS) cells at passage 4 and 9 using Life Technologies Click-iT@Plus EdU Alexa Fluor® flow cytometry assay kit. N=4 for p4 & N=3 for p9, all in technical triplicate. (C and D) Representative two-dimensional flow plots for each passage showing gating strategy to determine % of cells in each cell cycle phase for (B). (E) Clonal assay strategy to determine multipotency of self-renewing MesoT (FBS). (F) FACS gating strategy to obtain single cells for clonal analysis. Triple positive single cells (CD44⁺/CD73⁺/CD105⁺) were sorted onto a 96 well plate for amplification and downstream lineage analysis. Cyan are isotype controls. (G) Ater amplification, 14 individual clones were selected for downstream lineage analysis. MesoT (FBS) cells were treated with 2% FBS+VEGF or +PDGF-BB for endothelial or smooth muscle cell differentiation, respectively. Cells were fixed and probed with antibodies against vWF (endothelium) or MYH11 (SMC). >45 cells in 3 separate images per clone were quantified using ImageJ to determine % of cells that give rise to each lineage. (1) MesoT in CDM-Activin A treated with VEGF generate mixtures of endothelial cells (vWF⁺) and SMCs (calponin⁺). Scale bar, 50 μm. (I and J) Cells as in (H) were cultured on Matrigel for 12 days. Bright field images (I) of resulting vessel structures were probed with antibodies for αSMA and vWF and the nuclei counter stained with DAPI (J). Scale bars, 50 μm. Error bars±SEM. See also FIG. S4.

FIG. M5 shows that MesoT cells incorporate into newly formed blood vessels in a neonatal mouse heart injury model. (A and B) Surgery on P0.5 mouse pups that includes ventricle apex resection. After resection, the damaged heart was overlaid with a 2 μl suspension of 1×10⁶ DiO-labeled MesoTs (B) followed by suturing of the rib cage and chest wall. Micron bar, 1 mm. (C) Cryo-section (10 μm) of a repaired mouse heart, 30 days post-injury. Tissue was probed with antibodies for αSMA, CD31, human Golgi antigen, and DAPI. Scale bars, 50 μm. (D) Repair zone showing a submesothelial blood vessel (by) comprised of human αSMA⁺ cells and human CD31⁺ cells. Scale bars; top left, 20 μm; and other panels, 10 μm. See also FIG. S5. (E) Magnified images of panels shown in (E) showing incorporation of human endothelial and smooth muscle cells into nascent vessels. Scale bar, 10 μm.

FIG. M6 shows that reperfusion of a decellularized biological scaffold with MesoT vascular progenitor cells repopulates the vascular network and forms functional invested vessels when transplanted in vivo. (A-C) Image of a decellularized rat jejunum and (B and C) injected with phenol red to contrast the vasculature before cell perfusion. (D) MesoTs were perfused through the arterial and venous cannulas with (+VEGF) media. MTT assay imaging depicts reseeded vasculature with metabolically active cells after 28 days. Right panel is blowup of inset on left. Red lines mark representative vessels of various sizes. Micron bar, 1 mm. (E) Vasculature derived by seeding MesoTs was stained human nuclear antigen (hNA) and DAPI. (F-H) Vessels of small, medium and large diameter as in (D) were stained with antibodies against CD31 or αSMA. Scale bars, 100 μm. (Vascular barrier integrity testing by perfusion with FITC-dextran. Left panel: bright field image before perfusion; Middle panel: intravital microscopy image of FITC-dextran retention after repeated perfusion and washing of the vascular network; Right panel: uptake of acetylated low-density lipoprotein (LDL, red). Nuclei were visualized with NuBlue™ Live ReadyProbes™. Scale bars, 100 m. (J) Light sheet microscopy image of vessel networks after fixation and staining with CD31 antibody. Scale bar, 400 μm. (K) Reseeded vascular constructs were transplanted into 8-week-old immunodeficient female rats and anastomosed (white arrow) with the host circulatory system. (L) Gross anatomical image of a transplanted graft after harvesting, showing the presence of host oxygenated blood and the absence of occlusion or leakage. (M-O) Harvested grafts stained with antibodies for CD31 (red) and αSMA (green). Blood vessels of various sizes show a lining of endothelium and smooth muscle cells. Scale bars, 100 μm. See also FIG. S6.

FIG. S1, which is related to FIG. M1 shows (A) hESC-derived (WA09) splanchnic mesoderm (SplM) cells generated after 4 days of culture in CDM supplemented with Wnt3a (25 ng/ml) and BMP4 (100 ng/ml) were fixed and stained with antibodies for ISL1, NKX2.5 and FLK1. Scale bars, 50 μm. (B) Immunofluorescence analysis of K3 hiPSCs and hESCs (TE03, WA07 and WA01) cultured and stained as in (A). Nuclei were counter stained with DAPI. Scale bars, 50 μm. (C) qRT-PCR data showing fold-change of transcript levels for pluripotency markers (NANOG, OCT4 and SOX2) in SplM and MesoTs relative to hESCs. TaqMan assays for each transcript were performed in technical triplicate and fold-change shown relative to hESCs (WA09) after normalization with 18S RNA. (D) Flow cytometry data of untreated hESCs (WA09), SplM, and MesoT showing the absence of the pluripotent marker SOX2 (left plot) and presence of lineage specific marker ISL1 (middle plot) in SplM. As cells transition to MesoT, ISL1 is downregulated (right plot). (E) Immunofluorescence of WA07, TE03 and K3 hiPSC lines after differentiation of SplM to MesoT followed by probing with WT1 and TBX18 antibodies. Nuclei were counter stained with DAPI. Scale bar, 50 μm. (F) Flow cytometry pseudocolor plot of MesoT cells probed with antibodies for WT1 and TBX18, (G) WA09-derived MesoT cells were fixed and probed with antibodies for lineage specific markers WT1 and TBX18. Nuclei were counterstained with DAPI. Right hand side is a magnification of the insets from left. Scale bars, 50 μm. Error bars±standard deviation.

FIG. S2, which is related to FIG. M1, shows (A) Schematic showing the progression of splanchnic mesoderm (SplM) to mesothelium-like cells (MLCs) and then MesoTs. Removal and addition of growth factors and inhibitors are indicated above the arrows for each stage. (B) qRT-PCR data showing fold-change of transcript levels for pluripotency (OCT4, NANOG), SplM (ISL1 and NKX2.5) and mesothelium (WT1, TBX18, TCF21 and MSLN). TaqMan assays for each transcript were performed in technical triplicate and fold-change shown relative to untreated hESCs (WA09) after normalization with 18S RNA. (C) Immunofluorescence analysis of MLCs and MesoTs directly derived from MLCs. After EMT induction of MLCs (A), cells become migratory but retain expression of mesothelial lineage markers. Cells were fixed and stained with antibodies against ISL1, NKX2.5, WT1, TBX18, E-cadherin (epithelial marker) and alpha smooth muscle actin (αSMA). Scale bars, 50 μm. Error bars±standard deviation.

FIG. S3, which is related to FIG. M2 shows (A) Heatmap showing the relationship between cell type-specific DNA methylation modules (WA09 hPSCs, hPSC-derived splanchnic mesoderm, MesoTs and hPSC-derived cardiomyocytes). Module 9 comprises 1846 cytosines and is characteristic of MesoTs. (B) Heatmap showing highly enriched H3K27ac (blue) lineage specific sites in hESC-derived cardiomyocytes (CM) and the absence of H3K4me1 in corresponding sites for MesoT. (C and D) Gene Ontology analysis of genes analyzed in FIG. M2D.

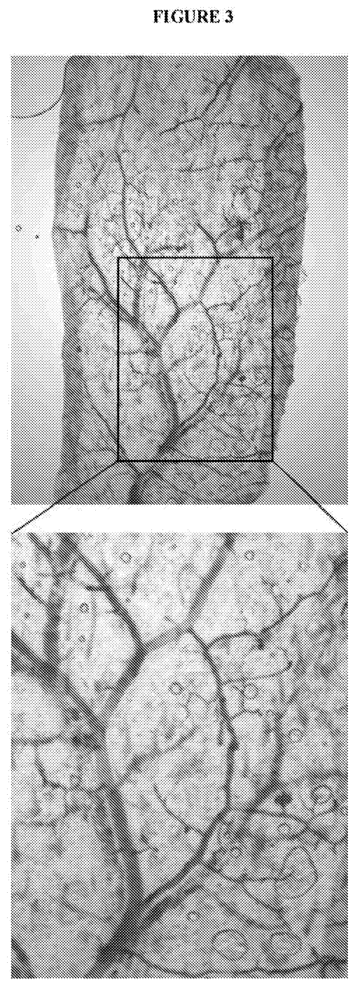

FIG. S4, which is related to FIGS. M3 and M4, shows (A) Cells numbers at different stages of differentiation. Cell number was counted at each stage after plating 1 million hESCs (WA09). (B) Immunofluorescence of MesoT cells probed with lineage specific antibodies for mesothelium (WT1), smooth muscle (calponin, MYH11), endothelium (vWF), or fibroblasts (DDR2). Nuclei were counterstained with DAPI. Scale bar, 50 m. (C) MesoT-derived Fibroblasts on day 12 were probed with antibodies for WT1, DDR2, calponin and counterstained with DAPI. (D) Contraction assays as for FIG. 3 except performed on WA09 hESCs. Contraction is shown as the % change in cell surface area for individual cells. Each treatment group was compared to corresponding control time point to determine statistical significance. N=20. (E) Flow cytometry histograms of MesoT cells probed with antibodies for CD31 (endothelium), NG2 (pericyte) and isotype control. (F) Immunofluorescence of MesoT (FBS) cells after culturing with 2% FBS+VEGF (endothelium) or +PDGF-BB (SMC). Cells were fixed and probed with antibodies against vWF or MYH11 and counter stained with DAPI. Scale bars, 50 μm. (G) Magnified images shown in FIG. 3J.

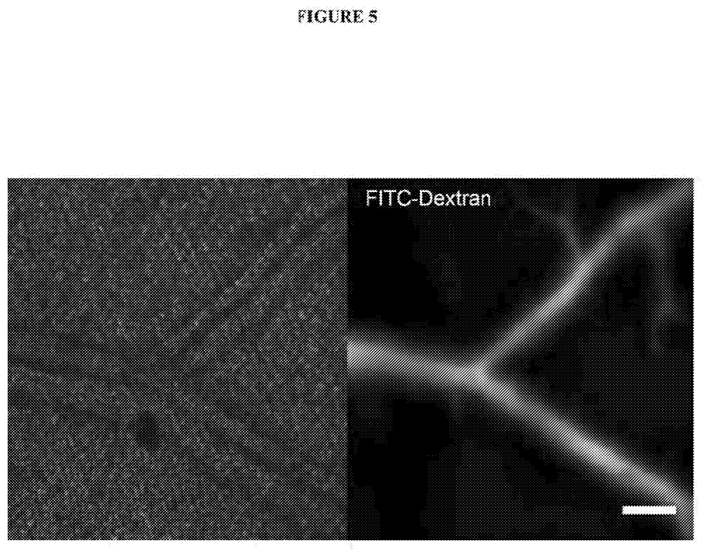

FIG. S5, which is related to FIG. M5 shows (A) Whole mount image of a mechanically injured heart 5 days after resecting part of the ventricle. (B) DiO labeled MesoT human cells (green) were applied immediately after resection and attached to injured area. Micron bar, 1 mm. (C) Immunohistochemistry image showing the absence of hGolgi⁺ cells in the repair zone of neonatal hearts that did not receive MesoT cells following mechanical injury. The section was also probed with antibodies for α-smooth muscle actin (αSMA), CD31 and counterstained with DAPI. Scale bar, 50 μm. (D) Blood vessel from FIG. 5D showing the presence of erythrocytes due to autofluorescence. Scale bar, 20 sm.

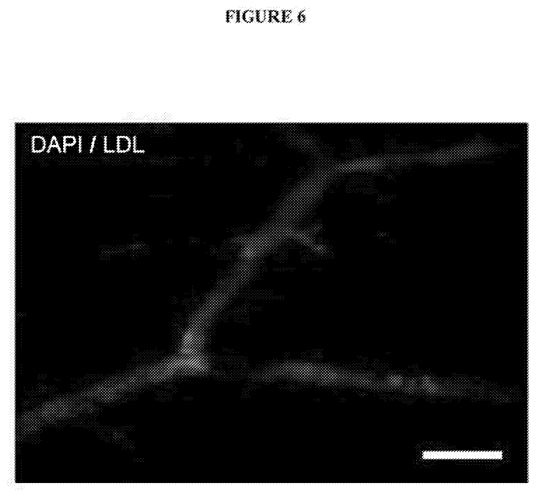

FIG. S6, which is related to FIG. M6, shows (A) Basic bioreactor design showing media vessels, pump, pressure sensors, and the inflow/outflow ports that circulate perfused media through the vascular bed. (B) MTT assay of a decellularized construct showing the absence of viable cells. (C) Left panel; bright field image before FITC-dextran perfusion. Time lapse images 5 and 25 seconds after FITC-dextran perfusion into decellularized scaffolds seeded at low density with MesoTs. Scale bar, 100 μm. (D and E) Light sheet microscopy images as in FIG. 6J showing CD31⁺ endothelium lining recellularized jejunal scaffolds after 28 days. Scale bars, 500 μm. (F) Light sheet microscopy image showing NG2⁺ pericytes lining MesoT-seeded vessels. Scale bar, 150 μm. (G) Gross anatomy image as in FIG. 6L after harvesting of anastomosed tissue.

FIG. S7, Table S1 shows a list of antibodies which are related to the experimental methods which are described herein.

FIG. S8, Table S2 shows Taqman Primers for qRT-PCR which are related to the experimental methods which are described herein.

### DETAILED DESCRIPTION OF THE INVENTION

The following terms shall be used to describe the present invention.

Unless otherwise noted, the terms used herein are to be understood according to conventional usage by those of ordinary skill in the relevant art (“the skilled practitioner”).

In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present vascular constructs and methods are disclosed and described, it is to be understood that this invention is not limited to specific conditions, or specific methods, or specific vascular constructs, etc., as such may, of course, vary, and the numerous modifications and variations therein will be apparent to those skilled in the art.

Standard techniques for growing and differentiating cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “patient” or “subject” is used throughout the specification within context to describe an animal, generally a mammal and preferably a human, to whom treatment, including prophylaxis, with the vascular constructs which are provided according to the present invention are provided. For treatment of those infections, conditions and/or disease states which are specific for a specific animal such as a human patient, the term patient refers to that specific animal.

The term “effective” is used to describe an amount of a component, compound or compositions which is used or is included in context in an amount and/or for a period of time (including sequential times) sufficient to produce an intended effect, principally the seeding and differentiation of MesoTs/EPCs in associate with tubular biomaterial scaffolds as described herein. By way of example, an effective amount of a differentiation agent is that amount which, in combination with other components, in a differentiation medium for an appropriate period of time (including sequential times when different differentiation agents are exposed to cells to be differentiated) will produce the differentiated cells desired.

As used herein, the term “activate” refers to an increase in expression of a marker or an upregulation of the activity of a protein or a marker associated with a cell, including endothelial cells, smooth muscle cells and pericytes which comprise the vascular tissue associated with the biomaterials scaffold to provide vascular vessels according to the present invention. These cells have utility as transplantable vasculature for use in vascular engineering of organs including heart, kidney, brain/CNS and pancreas, for surgical repair and/or replacement of blood vessels, the treatment and/or repair of vascular disease and degeneration, for treatment of patients with peripheral vascular disease secondary to diabetes, vascular occlusions (atherosclerotic), aneurysms, and in tissue reconstruction, following traumatic injury or surgery, among others as described herein.

As used herein when referring to a cell, cell line, cell culture or a population of cells, the term “isolated” refers to the cells described being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

As used herein, the term “express” refers to the transcription of a polynucleotide or translation of a polypeptide (including a marker) in a cell, such that levels of the molecule are measurably higher in or on a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “markers” or “biomarkers” describe nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

As used herein, the term “contacting” (i.e., contacting a cell with a compound or composition) is intended to include perfusing and/or incubating the compound and the cell together in vitro (e.g., adding a compound or composition to cells in culture). The term “contacting” is not intended to include the in vivo exposure of (“exposing”) cells to a differentiation agent that may occur naturally in a subject (i.e., exposure that may occur as a result of a natural physiological process). The step of contacting the cell with differentiation medium as otherwise described herein can be conducted in any suitable manner, but preferably the differentiation medium is perfused through the cells which are on the surface of the tubular biomaterials scaffold. The perfusion of the cells preferably takes place in a bioreactor which controls temperature, CO₂ concentration and humidity, among other factors.

The terms “treat”, “treating”, and “treatment” etc., as used herein, refer to any action providing a benefit to a patient at risk for or afflicted by a disease state, condition or deficiency which may be improved using cellular compositions according to the present invention. Treating a condition includes improving the condition through lessening or suppression of at least one symptom, delay in progression of the effects of the disease state or condition, including the prevention or delay in the onset of effects of the disease state or condition, etc. Treatment, as used herein, encompasses therapeutic treatment and prophylaxis encompasses prophylactic approaches for reducing the likelihood of a disease state and/or condition from occurring.

The term “human pluripotent stem cell-derived vascular progenitor (MesoT) cell of the mesothelium lineage”, migratory mesoderm cells, “MesoT cells” “MesoTs” or “MesoTs/EPCs” “epicardial progenitor cells”, “epicardial pluripotent cells”, “EPCs”, are synonymous terms which are used in the present invention to refer to the multipotent cells which are produced from human pluripotent cells (hPCs), including hESCs or from Is11+pluripotent cells (IMPs, also referred to herein as splanchnic mesoderm cells or SplM) by exposing hPCs to conditions which produce IMPs/SplM, and then exposing the resulting IMPs/SplM to conditions which produce MesoTs/EPCs. Although non-human MesoTs/EPCs can be used in veterinary aspects of the present invention, the preferred embodiments relate to the differentiation of human MesoTs/EPCs. This process to provide these cells is described in great detail in international application no. PCT/US2009/004334, published as WO 2010/011352, Jan. 28, 2010, in United States patent publication no. US2011-0305672, published Dec. 15, 2011 and U.S. Pat. No. 9,732,322, issued Aug. 15, 2017, all of which references being incorporated in their entirety herein. As indicated in these references, MesoTs/EPCs are produced by exposing IMPs/SplM in a differentiation medium in the presence of effective amounts of a GSK inhibitor (e.g., a Wnt protein such as Wnt3a as otherwise described herein or a GSK inhibitor such as BIO), a bone morphogenic protein (e.g., BMP4) and retinoic acid (preferably, all-trans retinoic acid) for a period of time sufficient to convert the IMPs/SplM to EPC/MesoTs (e.g., about 8 to 20 days or more, about 10 to 18 days, about 15-17 days or more). EPC/MesoTs may be produced directly from hPCs (described below) by exposing the cells initially to effective amounts of a GSK inhibitor (e.g., WNT3a or BIO), a bone morphogenic protein (e.g. BMP4) and optionally an Activin A inhibitor (e.g., SB431542) and then (generally, after about 2-8 days) further exposing the intermediate cells produced (which are IMPs/SplM) to the same conditions for converting IMPs/SplM to EPC/MesoTs as presented above (e.g., Wnt3a or BIO, BMP4 and all-trans retinoic acid for a period up to about 16-20 days or more). MesoTs/EPCs which are used in the present invention are preferably syngeneic or allogeneic to the patient.

MesoTs/EPCs are characterized by their ability to spread over the surface of a biomaterial scaffold forming an outer later and also by their capacity to migrate into the scaffold and form vascular tissue. A standard assay to evaluate the migratory properties of MesoTs/EPCs is to plate cells on a collagen I matrix.

Microarray analysis of MesoTs generated from three hESC lines and a human iPSC line indicates that MesoT cells express Wilm's tumor suppressor protein 1 (Wt1), Tcf21 (epicardin), Raldh2 (Aldhla2). These transcripts/biomarkers are primary identifiers of MesoTs/EPCs, a pro-epicardial-epicardial/vascular cell type generated from pluripotent cells in culture.

In addition to the above, MesoTs/EPCs also can express one or more (2, 3, 4, or 5) of Tbx18, COL3A1, GATA6, Tbx3 and Tbx5. A table summarizing some of the most up-regulated genes is shown in FIG. 47, Table 2 of PCT publication no. WO2010/011352.

MesoTs/EPCs may be further differentiated pursuant to the present invention in a differentiation medium as described herein for a period of at least 9-10 days, or at least 12-20 days, which period serves to differentiate the MesoTs/EPCs into a combination/mixture of endothelial cells and smooth muscle cells and most often pericytes which cells self-assemble into vascular networks when associated with a biomaterial scaffold which provides support for the tissue which is produced. The differentiation medium is preferably a chemically defined medium (CDM) which excludes Activin A and includes an effective amount of a VEGF agonist (of the VEGF pathway or VEGF receptor). Other media may also be used and include effective amounts of heregulin (e.g. approximately 5-25 ng/m, preferably 10 ng/ml), IGF (e.g. IGF-1 at 50-500 ng/m, preferably 200 ng/ml), FGF (e.g. FGF or FGF2 at 2-20 ng/ml, preferably 8-10 ng/ml) and a VEGF agonist (5-500 ng/ml, preferably 25-75 ng/ml, most often 50 ng/ml of rhVEGF-A₁₆₃), in the absence of Activin A.

The term “primate Pluripotent Stem Cells”, of which “human Embryonic Stem Cells” or hESCs and human induced pluripotent stem cells or hiPSCs are a subset, are derived from pre-embryonic, embryonic, fetal tissue or adult stem cells (in the case of human induced pluripotent stem cells) at any time after fertilization, and have the characteristic of being capable under appropriate conditions of producing progeny of several different cell types that are derivatives of all of the three germinal layers (endoderm, mesoderm and ectoderm), according to a standard art-accepted test, such as the ability to form teratomas in 8-12 week old SCID mice. The term includes both established lines of stem cells of various kinds, and cells obtained from primary tissue that are pluripotent in the manner described.

Included in the definition of pluripotent or pPS cells (pPSCs) are embryonic cells of various types, especially including human embryonic stem cells (hESCs), described by Thomson et al. (Science 282: 1145, 1998); as well as embryonic stem cells from other primates, such as Rhesus stem cells (Thomson et al., Proc. Natl Acad. Sci. USA 92: 7844, 1995). Other types of pluripotent cells are also included in the term. Human Pluripotent Stem Cells includes stem cells which may be obtained from human umbilical cord or placental blood as well as human placental tissue. Any cells of primate origin that are capable of producing progeny that are derivatives of all three germinal layers are included, regardless of whether they were derived from embryonic tissue, fetal, or other sources. The pPS cells are preferably not derived from a malignant source. It is desirable (but not always necessary) that the cells be karyotypically normal.

pPS cell cultures are described as “undifferentiated” when a substantial proportion of stem cells and their derivatives in the population display morphological characteristics of undifferentiated cells, clearly distinguishing them from differentiated cells of embryo or adult origin. Undifferentiated pPS cells are easily recognized by those skilled in the art, and typically appear in the two dimensions of a microscopic view in colonies of cells with high nuclear/cytoplasmic ratios and prominent nucleoli. It is understood that colonies of undifferentiated cells in the population will oflen be surrounded by neighboring cells that are differentiated.

Pluripotent stem cells may express one or more of the stage-specific embryonic antigens (SSEA) 3 and 4, and markers detectable using antibodies designated Tra-1-60 and Tra-1-81 (Thomson et al., Science 282:1145, 1998). Differentiation of pluripotent stem cells in vitro results in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde, and then developing with Vector Red as a substrate, as described by the manufacturer (Vector Laboratories, Burlingame Calif.) Undifferentiated pluripotent stem cells also typically express Oct-4 and TERT, as detected by RT-PCR.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered.

The types of pluripotent stem cells that may be used to produce MesoTs/EPCs useful in the present invention include established lines of pluripotent cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples are established lines of human embryonic stem cells or human embryonic germ cells, such as, for example the human embryonic stem cell lines WA01, WA07, and WA099 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG00v (BresaGen, Athens, Ga.), as well as normal human embryonic stem cell lines such as WA01, WA07, WA09 (WiCell) and BG01, BG02 (BresaGen, Athens, Ga.). All of the stem cells which may be used to produce MesoTs/EPCs pursuant to the present invention are obtained ethically, through established ethical guidelines.

Epiblast stem cells (EpiScs) and induced pluripotent stem cells (iPSCs), especially human induced pluripotent stem cells (hiPSCs) fall within the broad definition of pluripotent cells which may be used to provide EPCs.

Human embryonic stem cells (hESCs) may be prepared by methods which are described in the present invention as well as in the art as described for example, by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 199).

The term “embryonic stem cell” refers to pluripotent cells, preferably of primates, most often humans, which are isolated from the blastocyst stage embryo. Human embryonic stem cell refers to a stem cell from a human and are preferably used in aspects of the present invention which relate to human therapy or diagnosis. The following phenotypic markers are expressed by human embryonic stem cells:

SSEA-3, SSEA-4, TRA-1-60, TRA-1-81, CD9, alkaline phosphatase, Oct 4, Nanog, Rex 1, Sox2 and TERT. See Ginis, et al., Dev. Biol, 269(2), 360-380 (2004); Draper, et al., J. Anat., 200 (Pt. 3), 249-258, (2002); Carpenter, et al., Cloning Stem Cells, 5(1), 79-88 (2003); Cooper, et al., J. Anat., 200 (Pt. 3), 259-265 (2002); Oka, et al., Mol. Biol. Cell, 13(4), 1274-81 (2002); and Carpenter, et al., Dev. Dn., 229(2), 243-258 (2004). Any primate pluripotent stem cells (pPSCs), including especially human embryonic stem cells or human induced pluripotent stem cells can be used to produce mesoderm Is11+(IMP) cells or multipotent epicardial progenitor cells (EPCs) according to the present invention, preferred pPSCs for use in the present invention include human embryonic stem cells and especially human induced pluripotent stem cells.

The term “differentiation” is used to describe a process wherein a MesoT cell/EPC acquires the features of a more specialized cell such as, for example, an endothelial cell, a smooth muscle cell and in many instances a pericyte, which will self-assemble into vascular tissue associated with a biomaterials scaffold. The term “differentiated” also includes the process wherein a multipotent stem cell, including a hESC, becomes a more specialized intermediate cell such as a progenitor cell (a Splanchnic cell SplM or IMP) or a SplM/IMP cell becomes a MesoT/EPC. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

Compositions which may be used for therapies as described above include an effective amount of MesoTs/EPCs for carrying out the therapy, i.e., by seeding cells onto the surface of a biomaterial scaffold and differentiating the MesoTs/EPCs into several vascular lineages (at least endothelial cells and smooth muscle cells, but in most instances the endothelial cells and smooth muscle cells also will include pericyte cells) which self-assemble into cellular vascular tissue which is supported by the biomaterial scaffold. In instances where a preferred biomaterial is formed from decellularized tubular tissue obtained from an animal (often a human, non-human primate, pig or cow), the MesoTs are often deposited through the pedicles of the biomaterial and into the vascular tree which exists in these decellularized animal tissue. The composition comprises between 5×10⁴ and 5×10⁹ cells, often between 10⁵ and 10⁸, more often between 10⁵ and 10⁶ cells suspended in differentiation medium or saline (preferably differentiation medium) as otherwise described herein. The amount of differentiation or saline solution generally ranges from about 50 ul to about 10 ml or more, preferably about 100 ul to about 2 ml. The composition may be deposited directly onto the scaffold (on both the inside and outside surfaces), but preferably, the cells are deposited onto the scaffold within the internal structure of the biomaterial in order to allow growth of the cells within the biomaterial. The cells may be administered in the absence of bioactive agents or including bioactive agents, which may be included to effect a pharmacological result associated with the repair or treatment of the vascular structure in the patient to be treated.

As used herein, the terms “differentiation medium” and “cell differentiation medium” are used to describe a cellular growth medium in which (depending upon the additional components used) the hESCs, splanchnic mesoderm cells (Is11+multipotent cells or IMPs) or preferably, migratory mesoderm cells (MesoTs) or epicardial progenitor cells (EPC's) or other cells are differentiated into more mature cells, in preferred embodiments, a combination of endothelial cells, smooth muscle cells and perciytes which self-assemble into vascular tissue (e.g. a vascular vessel) associated with a scaffold, providing a vessel which can serve as a natural blood vessel. “Vascular differentiation medium” and “seeding medium” are media which are used to seed and differentiate MesoTs into at least endothelial cells and smooth muscle cells, more often endothelial cells, smooth muscle cells and pericytes which over a period of differentiation of at least 10-12 days self-assemble in association with a biomaterial scaffold to produce vascular vessels according to the present invention. Specific examples of these are presented in the examples section and otherwise as follows. Differentiation media are well known in the art and comprise at least a minimum essential medium plus one or more optional components such as serum albumin (bovine serum albumin or a fraction thereof) non-essential amino acids, antibiotics (e.g., penicillin, streptomycin, mixtures thereof, etc.), salts (including trace elements), transferrin, beta mercaptoethanol, ascorbic acid, growth factors such as fibroblast growth factor (FGF 1 and/or especially FGF2), insulin-like growth factors (e.g., LR-IGF, native IGF), insulin, heregulin (especially heregulin β) and an agonist of VEGF signaling pathway or VEGF receptor, including VEGF and/or a VEGF agonist (especially rhVEGF-A₁₆₅, a small molecule VEGF agonist or a protein agonist such as gremlin, among others) glucose, glutamine, and other agents well known in the art and as otherwise described herein. In preferred embodiments, the vascular differentiation medium (i.e. the medium which is used to seed and/or differentiate the MesoTs to produce vascular tissue) is chemically defined media (CDM) which excludes Activin A and includes an effective amount of an agonist of VEGF signaling pathway or VEGF receptor, including VEGF and/or a VEGF agonist, preferably rhVEGF-A₁₆₅.

Media for differentiating types of cells, including MesoTs/EPCs may also include basal cell media which contains between 1% and 20% (preferably, about 2-10%) fetal calf serum, or for defined medium (preferred) an absence of fetal calf serum and KSR, and optionally including bovine serum albumin (about 1-5%, preferably about 2%). Preferred differentiation medium is defined (e.g. chemically defined media CDM) and is serum free. In the case of amplification medium in which a population of MesoTs is amplified (e.g. clonal amplification) in order to provide sufficient numbers of cells for seeding of biomaterial foundations/scaffolds and formation of vascular tissue, the preferred media is CDM, which is supplemented with fetal bovine serum (e.g. 10% FBS) to support maintenance and proliferation of MesoT cells over an amplification timeframe to obtain an appropriate number of MesoT cells for seeding of biomaterials foundations and/or supports and differentiation.

Other agents which optionally may be added to differentiation medium depend on the cells to be differentiated and include, for example, nicotinamide, members of TGF-β family, including TGF-β1, 2, and 3, nodal, serum albumin, members of the fibroblast growth factor (FGF) family, platelet-derived growth factor-AA, and -BB, platelet rich plasma, insulin growth factor (IGF-L 1, LR-IGF), growth differentiation factor (GDF-5, -6, -8, -10, 11), glucagon like peptide-I and II (GLP-1 and H), GLP-1 and GLP-2 mimetobody, Exendin-4, parathyroid hormone, insulin, progesterone, aprotinin, hydrocortisone, ethanolamine, epidermal growth factor (EGF), gastrin I and II, copper chelators such as, for example, triethylene pentamine, forskolin, Na-Butyrate, betacellulin, ITS, noggin, neurite growth factor, nodal, valporic acid, trichostatin A, sodium butyrate, hepatocyte growth factor (HGF), sphingosine-1, VEGF, MG132 (EMD, CA), N2 and B27 supplements (Gibco, Calif.), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, heregulin, or combinations thereof, among a number of other components. Each of these components, when included, are included in effective amounts in order to facilitate the differentiation of a cell into a mature differentiated cell. In the case of MesoT differentiation to produce endothelial cells, smooth muscle cells, most often including pericytes, the differentiation medium contains effective amounts of heregeulin, IGF, FGF (e.g., FGF2) and a VEGF agonist, but excludes Activin A. A preferred medium containing these components is CDM minus Activin A, as described in greater detail herein.

Byway of further example, suitable media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12150% DMEM basal medium; 200 mM L-glutamine, Gibco #15039-027; non-essential amino acid solution, Gibco 11140-050; R-mercaptoethanol, Sigma #M7522; human recombinant basic fibroblast growth factor (bFGF), Gibco #13256-029. Preferred embodiments of media used in the present invention include chemically defined media (CDM) which excludes Activin A and are as otherwise described herein. The preferred medium for differentiating MesoTs pursuant to the present invention includes DMEM/F12 or CDM (chemically defined media), with added components as otherwise described herein. CDM is the more preferred media with added components (heregulin, IGF, FGF and VEGF agonist) for differentiating MesoTs into endoethelial, smooth muscle cells, mixtures of these cells, further including pericytes which unexpectedly self-assemble into vascular tissue.

MesoTs may be seeded onto a vascular matrix scaffold in seeding media such as DMEM/F12 or CDM, supplemented with heregulin β, IGF, FGF2 and an agonist of VEGF signaling pathway or VEGF receptor (VEGFR) agonist, such as VEGF (including VEGF small molecule agonist or protein—gremlin), among other components.

### Exemplary Seeding Media Formulation for Cell Seeding and Differentiation

### DMEM/F12 (Mediatech)

2% probumin (bovine serum albumin fraction V) biotech grade (Millipore)
1×nonessential amino acids
50 U/mL penicillin
50 μg/mL streptomycin
1×trace elements A,B,C (Mediatech)
10 μg/mL transferrin (Athens Research & Technology)
0.1 mM β-mercaptoethanol (Gibco)

50 μg/mL ascorbic acid (Sigma)

8 ng/mL FGF2 (Invitrogen) 0.01 ng/ml-1 microg/ml
200 ng/mL LR-IGF (Sigma) 0.2 ng/ml-20 microg/ml or a native IGF (0.2 ng/ml-20 microg/ml) or insulin (0.2 ng/ml-100 microg/ml)
10 ng/mL heregulin β (Peprotech) 0.01 ng/ml-1 microg/ml
10 ng/ml rhVEGF-A₁₆₅ (R&D Systems) 0.01 ng/ml-1 microg/ml,
Or, a VEGF signaling pathway agonist or a VEGFR agonist such as a small molecule or a protein agonist such as Gremlin may be substituted for the VEGF factor or added to the medium so that several VEGF components are included in the differentiation medium, which produces a mixture of endothelial cells, smooth muscle cells and often pericytes (in in vitro systems the pericytes may not be produced, but when the cells are differentiated within a biomaterials foundation or support, will often appear in the presence of endothelial cells and smooth muscle cells. The media used to differentiate MesoTs/EPCs into endothelial, smooth muscle cells and optionally, pericytes is Activin A free and includes heregulin, IGF FGF2 and a VEGF agonist component as described above, preferably VEGF-A₁₆₅. In the case of endothelial cells, the VEGF components are included in the media (e.g. VEGF-A₁₆₅ at a concentration of 20-100 ng/ml, 30-70 ng/ml, preferably 50 ng/ml), as well as, an inhibitor such as SB431542 (a TGF/Activin/Nodal pathway inhibitor at a concentration of 10-30 μM, preferably 20 μM). In the case of smooth muscle cells, these are produced using the same media as above (without the VEGF) but including platelet derived growth factor BB PDGF-BB (e.g. 20-100 ng/ml, 30-70 ng/ml, preferably 50 ng/ml PDGF-BB). To produce fibroblasts, the PDGF-BB is substituted with PDGF-AA at the same concentrations of PDGF-BB. To generate a mixture of endothelial cells and smooth muscle cells (which often will include pericytes), the media described above (which does not contain Activin A) will include an effective amount of the VEGF component (VEGF agonist). The mixture of cells self-assembles during differentiation to produce vascular tissue.

Preferred media for differentiating MesoTs/EPCs after seeding on/into biomaterials foundation or support to produce endothelial cells, smooth muscle cells and most often pericytes which self-assemble into vascular tissue is chemically defined media (CDM) which excludes Activin A and includes a VEGF agonist, although numerous media for growing cells known in the literature for example, as described above also may be used (all of which should preferably include an effective amount of heregulin, IGF, FGF2 and a VEGF agonist), CDM media consists of DMEM/F12 w/o glutamine supplemented with 1× nonessential amino acids (Corning, 25-25-CI), 1× antimycotic/antibiotic (Corning, 30-004-CI), 1× trace elements A (Corning, 25-021-CI), 1× trace elements B (Corning, 25-022-CI), 1× trace elements C (Coring, 25-023-CI), 2 mM L-alanyl-L-glutamine (Corning, 25-015-CI), 10 μg/ml transferrin (HOLO), human plasma, tissue culture grade (Athens Research and Technology, 16-16-032001-LEL), 2% Probumin® bovine serum albumin life science grade (EMD Millipore, 821005), 0.1 mM β-mercaptoethanol (Gibco, 21985023), 50 μg/mL ascorbic acid (Sigma-Aldrich, A8960), 10 ng/ml rhHeregulin β-1 (Peprotech, AF-100-03), 200 ng/ml LONG® R3 IGF-I human (Sigma-Aldrich, 85580C), 10 ng/ml rhActivin A (R&D Systems. 338-AC) and 8 ng/ml rhFGF basic (R&D Systems, 4114-TC). The preferred media which is used for differentiation is the above media (CDM) which excludes Activin A and includes an effective amount of a VEGF agonist (which can be a VEGF pathway or receptor agonist).

As discussed above, MesoTs/EPCs may be prepared by the methods as described above or as otherwise known in the art. From MesoTs/EPCs, endothelial cells, smooth muscle cells and/or pericytes each may be prepared alone or as mixtures (e.g., endothelial cells and smooth muscle cells or endothelial cells, smooth muscle cells and pericytes) and these cells will self-assemble into vascular tissue during the differentiation process. Mixtures of these cells will self-assemble after a period of about 10-12 days (from initial seeding of the MesoTs/EPCs to about 15-20 days or longer).

Pursuant to the present invention, the MesoTs/EPCs are isolated, resuspended in seeding (differentiation) medium and applied to a vascular matrix scaffold, in preferred embodiments by perfusing the cells with seeding media into the vascular matrix. Depending on the matrix used to provide support for the growth and differentiation of cells, the cells are deposited (seeded) onto the surface of the matrix (often this steps takes place by perfusing the cells in seeding medium until they are deposited onto the surface of the matrix. The seeding process takes place over a number of days (e.g., each day a new bolus of EPC's in medium are perfused onto the surface of the matrix scaffold until maximum seeding takes place), often several days to a week or more. After seeding, the vascular scaffolds comprising the deposited EPC's are perfused with seeding media until vasculature has formed. Often this takes at least a week to 6 weeks or more, often 12-14 days to 30 days, depending on the size (diameter and length) of the vessel to be produced. Although the present invention may be readily used to produce vessels of varying lengths and diameters including vessels which are at least 5 mm or greater (e.g., up to 25 mm in diameter, preferably less than 20 centimeters in diameter, often less than 10 mm in diameter, often from 5 mm to 10 mm), in preferred aspects the present invention is particularly useful in producing vessels which are less than 5 mm in diameter, including vessels which are less than 4.5 mm, 4.25 mm, 4.0 mm, 3.75 mm, 3.5 mm, 3.25 mm, 3.0 mm, 2.75 mm, 2.5 mm, 2.25 mm, 2.0 mm, 1.75 mm, 1.5 mm, 1.25 mm, 1.0 mm, 0.75 mm, 0.5 mm, 0.25 mm or less in diameter. The length of the vessel can vary from less than a millimeter in length to several hundred centimeters in length, with most vessels having a length which ranges from 1.0 mm to about 15 centimeters in length.

The term “self-assembly” is used to describe the ability of MesoT derivative cells (i.e. endothelial, smooth muscle cells and in most instances pericytes), under the conditions of differentiation described herein, to be assembled in vessels (often tubular in nature) into vascular tissue comprising a distinct inner endothelial layer and an outer smooth muscle layer. This occurs due to the signaling conditions established by the media used in combination with the presence of a suitable scaffold to which cells assemble to form the vascular vessel structure (e.g. a vascular vessel).

The term “scaffold” or “scaffold matrix” is used to describe the support matrix upon which MesoTs according to the present invention are first seeded and differentiated into vascular tissue comprising endothelial cells, smooth muscle cells and pericytes which self-assemble into the vascular tissue. Scaffolds which are used in the present invention comprise biomaterials as foundations and/or supports which are fashioned (e.g., electrospun or 3D printed) into a tubular construct, each construct preferably having at least one portal or hole in the construct (for example, a pedicle in the case of decellularized animal vascular tissue or a portal or small hole in the case of a collagen or collagen/polymer mixture or other biomaterial as described herein) and in certain embodiments numerous portals in the tubular construct in order to facilitate perfusion of MesoTs for deposition onto the surface of the scaffold and differentiation by exposing the deposited cells to differentiation medium for a period of time sufficient to differentiate the MesoTs to final vascular tissue as otherwise described herein. Perfusion often takes place in a computer controlled bioreactor or other device (to control temperature, CO2 concentration and humidity) to facilitate perfusion of the differentiation medium in contact with the cells to be differentiated on the scaffold/matrix.

Scaffolds for use in the present invention include numerous materials (biocompatible materials or biomaterials), with a preferred scaffold being decellularized tubular tissue, especially vascular tissue, obtained from an animal, preferably a human, non-human primate, pig or cow (bovine). Decellurization of tubular animal tissue occurs using a combination of physical agitation, chemical surfactant/detergent treatment removal and enzymatic digestion disrupts cells and removes proteins and nucleic acids from the tubular tissue to provide a decellularized vascular scaffold. Numerous chemical and/or mechanical methods are known in the art to provide a decellularized scaffold, with certain methods utilizing continuous perfusion of solutions to effect mechanical removal of cell debris during chemical dissociation of the scaffold's cellular content. A general method to effect decellularization is to expose the tubular tissue obtained from the animal to a first step wherein the tissue is exposed to deionized water at low temperature (preferably at a temperature less than 10° C., more often less than 5° C.) for a period of several hours to a day or two to disrupt cells, followed by washing the tissue in PBS solution (for at least a few minutes to several hours or more, often about 30 minutes); then exposing the tissue to surfactant solutions such as a sodium deoxycholate solution (often a 4% solution) at room temperature for between one and eight hours (preferably, about 4 hours) to remove proteins and lipids followed by washing the tissue in PBS solution (for at least a few minutes to several hours or more, often about 30 minutes) and finally subjecting the tissue to enzymatic digestion to remove any remaining proteins, lipids and nucleic acids/nucleotides in the tissue by exposing the tissue to a solution comprising one or more enzymes such as DNase (e.g. 1 mg/ml of DNase 1) at room temperature for 1-12 hours (often 3 hours or overnight). This enzymatic digestion step is followed by washing the tissue with PBS solution for a few minutes to several hours or more, often about 30 minutes). The tissue obtained is then subjected to sterilization, preferably by exposing the tissue to gamma radiation (e.g., 100 Gy gamma irradiation) and is thereafter stored at low temperature (e.g. 2-8° C., often 4° C.) until use.

For example, one preferred approach to provide decellularized scaffold is to take tubular tissue obtained from an animal and expose the tissue first to deionized water for from several hours to a day or two at low temperature (e.g., 4° C.), followed by perfusion with 4% sodium deoxycholate at room temperature for 2-6 hours, often 4 hours) and finally with a DNAase (e.g., 1 mg/ml DNase-1 (Sigma) at room temperature for several hours (e.g. 1-5 hours, often 3 hours), with every step being followed by a 30 minute Phosphate Buffered Serum (PBS) wash. After chemical/mechanism treatment as described above, the material is washed for a final time, dried, stored and eventually used.

In addition to decellularized tubular tissue (often allogenic or xenogeneic), scaffolds may be created out of numerous materials including metallic biomaterials, including stainless steel, CoCr alloys, titanium alloys, Ti₆Al₄V, among others, ceramics, including those fashioned out of alumina, zirconia, carbon and hydroxyapatite, and polymeric materials, especially including polymeric materials which are mixed with collagen and electrospun and/or 3D printed. It is noted that the use of metallic and ceramic biomaterials, which tend not to be flexible are less preferred and such use is reserved for components which are rigid. Preferred polymers include ultra-high molecular weight polyethylene, polyurethane, polyesters such as poly(lactic-co-glycolic acid)(PLGA), polylactide (PLA), poly(giycerolsebacate)(PGS) and poly(caprolactone)(PCL), expanded polytetrafluoroethylene (ePTFE), polyethylene terephthalate, polyethylene glycol among others, each of which polymers may be used alone or blended with between 5% and 95% collagen (especially collagen I, which is a major extracellular matrix (ECM) component in natural blood vessels), preferably 5% to 75% collagen (5%, 10%, 15%, 20%, 25%, 30%, 35%, 40%, 45%, 50%, 55%, 60%, 65%, 70% or 75% collagen) by weight. Collagen, fibrin gels and fibers, fibronectin, proteoglycans, glycosaminoglycans and glycoproteins and other proteins may be particularly suited for use as biomaterials for vessels pursuant to the present invention. Many of the biomaterials used in vessels pursuant to the present invention contain biocompatible fillers and are nanocomposites. These materials are electrospun or 3D printed into tubular materials of varying diameters and lengths to reflect the desired size of the vessel after seeding and differentiation of the EPCs. In certain embodiments, biomaterials which are electrospun or 3D printed may have different materials or different blended materials in different layers of the scaffold. In certain embodiments this layering creates a laminate-like effect. These techniques are well known in the art. See, for example, Syedain, et al. Nature Communications 2017; Egger, et al., Bioengineering 2017 and Schürlein, et al., Biolechnology J., 2017 (for decellularized blood vessels); Hasan, et al. Acta Biomaterialia, 2013; Ahn, et al., 2015 Acta Biomaterialia; Catto, et al., Materials Science and Engineering, 2015; Radakovic, et al., PLOS One, 2017 (for electrospun vessels of various composition); Huang, et al., 2017, Biotechnology Journal 12; Leberfinger et al., 2017, Stem Cells Translational Medicine; and Kolesly, et al., PNAS 2016 (3D printed constructs of various characteristics. Additional references describing various biomaterials for vascular tissue engineering include Bertram, et al., BioMed Research International, 2017 and Ravi, et al., Regen. Med., 2010, January 5(1) 107.

Scaffolds according to the present invention preferably contain, in addition to an opening in the distil ends of the tubular material, additional small ports or portals which facilitate the seeding/perfusion of EPCs on the biomaterial surface and perfusion of the differentiation medium onto the cells on the biomaterial surface.

The present invention may be understood more readily by reference to the following detailed description of the preferred embodiments of the invention and the Examples included herein. However, before the present compositions and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

### EXAMPLES

### Graft/Scaffold Harvesting.

Lewis rats (age: 6-8 weeks; body weight 200 g) were obtained from Charles River and underwent scaffold harvesting under compliance with ethics review formalities. General anesthesia was induced by continuous isoflurane inhalation-anesthesia. The animals were sacrificed by an overdose of the anesthetic. A median laparotomy was used to isolate a 5-10 cm long segment of the jejunum, including its artery and vein pedicle. Following systemic administration of heparin (100 IE/kg) the feeding artery and draining vein were cannulated with a 20-Gauge catheter and flushed with PBS. Venous backflow was controlled macroscopically. A segment of a sacrificed rat's jejunum was explanted together with its cannulated feeding artery and draining vein. Residual blood inside the supplying vessels was flushed with PBS-Heparin (100 IE/kg) and the lumen of the segment was cleared of all feces. The specimen was stored at 4° C. until further processing.

### Matrix Preparation and Acellularization

To obtain a cell-free matrix the scaffold was decellularized using a modified method described by previously by DeCoppi. In brief, both, the intestinal lumen and the vascular tree were continuously perfused for mechanical removal of cell debris during chemical dissociation of the scaffold's cellular content. For decellularization, the scaffold was subsequently perfused with deionized water 4° C. for 24 h, followed by perfusion with 4% sodium deoxycholate (Sigma) at room temperature (RT) for 4 h, and finally with 1 mg/m DNase-I (Sigma) at RT for 3 h. Every step was followed by a 30 min PBS wash. After treatment, the constructs were sterilized by 100 Gy gamma irradiation and stored in PBS at 4° C.

Cellularization of Vasculature with EPCs

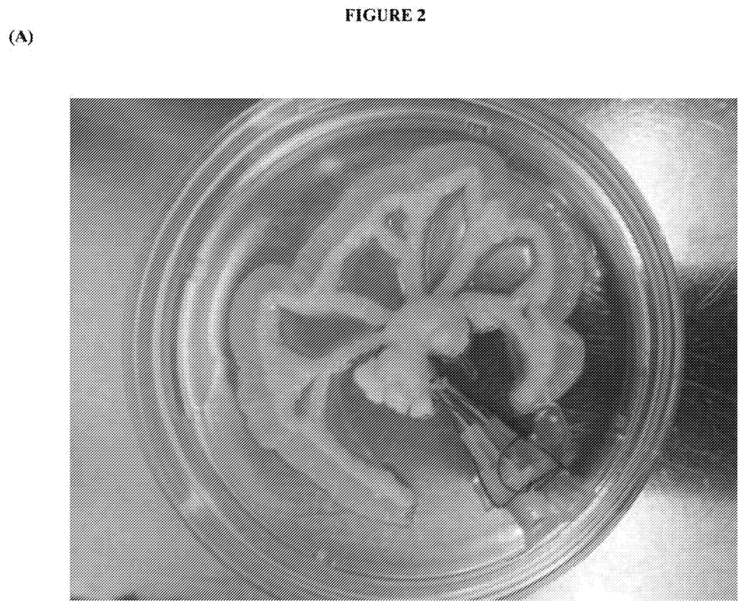

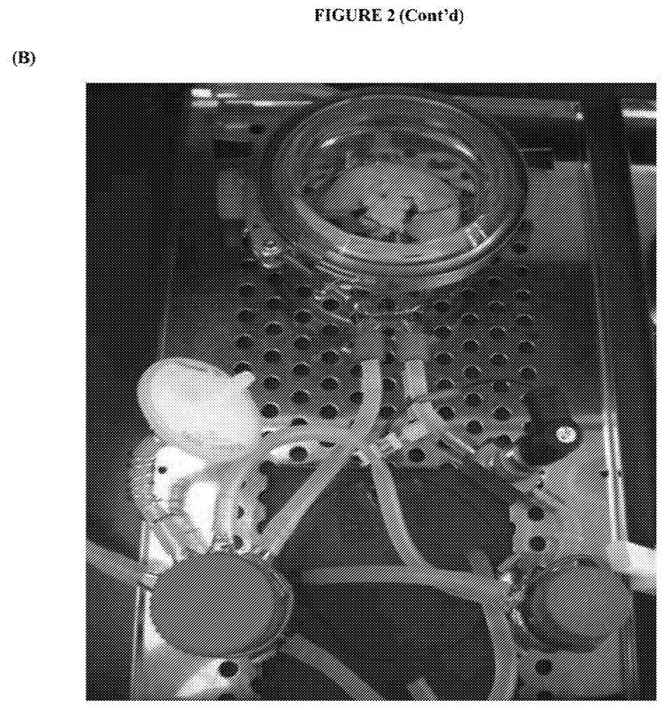

For recellularization, detached EPCs were injected through the pedicles of the scaffold into the vascular tree of the scaffold and per each day a new bolus of cells was perfused through the construct to maximize seeding and the cells were fused over 3 days. The media was DMEM/F12 supplemented with heregulin β, IGF, FGF2 and VEGF. To allow maturation and functional lining of the vascular bed the scaffold was connected to a bioreactor in which the vascular system was perfused dynamically. For standardization and reproducibility the perfusion in the bioreactor was computer controlled. Temperature, CO₂ concentration and humidity were monitored and adjusted as well as the dynamic perfusion was controlled in order to achieve physiological blood circulating conditions. After prevascularization of the vascular tree media was continually perfused for up to 28 days to allow maturation of endothelial, smooth muscle and pericyte layers. FIG. 2 shows the system used to evaluate vascular activity of EPCs in a decellularized vessel system. Top. Decellularized rat jejenum BioVac before perfusion with cells. Bottom. BioVac system enclosed within a temperature and gas-controlled incubator.

The decellularized BioVac construct obtained was seeded with EPCs and differentiated for 28 days after which the tissue was stained with MTT dye that recognizes viable cells (purple). FIG. 3 indicates that viable cells have lined the vasculature network. FIG. 3, top—shows the tissue at low magnification. FIG. 3, bottom—shows a higher magnification of the image directly above (as indicated by box). The conclusion drawn from this experiment and the data presented in FIG. 3 is that differentiated EPCs (as endothelial cells, smooth muscle cells and pericytes) successfully adhere to decellularized vascular tree and maintain viability.

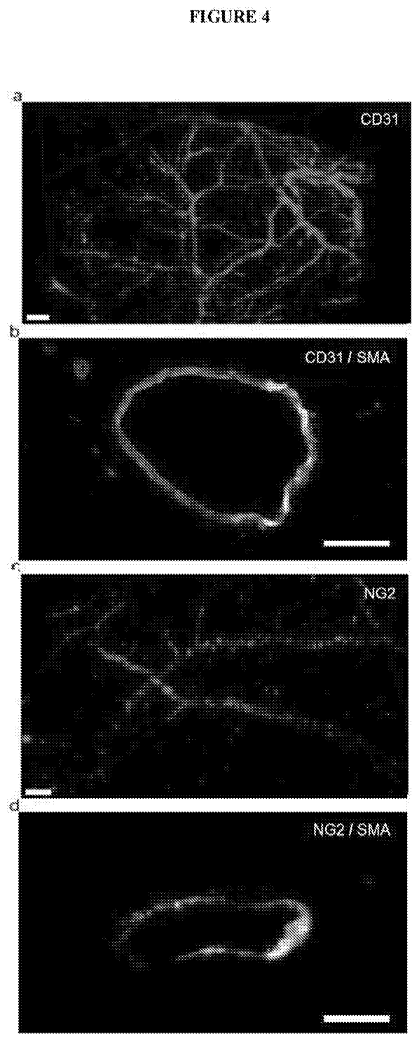

To determine cell viability live/dead staining with FDA (Fluorescein diacetate)/PI (Propidium iodide) (Sigma) was used for determining cell viability/cytotoxicity within the scaffolds. Briefly, cell seeded scaffolds were washed with PBS. Subsequently, a PBS assay solution containing 0.5 μg/ml FDA and 0.45 μg/ml PI was added to the samples incubated at room temperature for 10 sec and immediately evaluated using a fluorescence microscope (Keyence BZ-9000). The EPCs line the vascular tree in decellularized constructs at high efficiency, differentiate into multiple vascular lineages and self-assemble into a mature vessel structure lined on the luminal surface by CD31+endothelial cells and on the outer surface by SMA+smooth muscle cells and NG2+pericytes. This is shown in FIG. 4.

### Whole-Mount Staining for Light Sheet Fluorescence Microscopy (LSFM).

After fixation, the sample and the whole scaffold was immunofluorescence co-stained as stated above and an optical clearing was performed. The clearing was a two-step process involving a 2 h incubation in n-hexane followed by three times 30 min incubation in benzyl benzoate/benzyl alcohol (⅓ v/v).

Immunofluorescence Immunohistochemical stainings were performed to validate the scaffold's functional integrity after re-endothelialization. In brief, after explanation the scaffold was fixed in 4% paraformaldehyde at room temperature for 2 h and subsequently embedded in paraffin. 5 μm sections were deparaffinized by dehydration and antigens retrieved as per primary antibodies manufacturer's protocol. The primary antibody was incubated over night at 4° C. The secondary antibody conjugated with fluorochrome was incubated for 1 h at room temperature. Washing steps were conducted subsequently after each step. All fluorescence imaging was performed on a Keyence BZ-9000 fluorescence microscope. The used antibodies were: CD31, NG2, αSMA (abcam), Alexa Fluor 488 FITC, Alexa Fluor 555, Alexa Fluor 647 (Invitrogen). FIG. 4 shows the light sheet microscopy of recellularized vascular tree subject to IF analysis by probing with (a) CD31 antibody (scale bar 20 microns), (b) CD31 and SMA (scale bar, 25 microns), (c) NG2 (scale bar, 150 microns) and (d) NG2 and SMA (scale bar, 25 microns). Vessels formed by seeding the decellularized vascular tree retain FITC-dextran and do not leak. This experiment and results generated demonstrate the functional integrity of the vessels formed with EPCs.

### Intravital Microscopy

Intravital microcopy was performed to validate the scaffold's functional integrity after re-vascularization. For intravital microscopy the graft was placed under a standard inverted microscope (Zeiss) and perfused with carbogen-gassed PBS solution at 37° C. To analyze vessel integrity and microvascular permeability real-time fluorescence was detected after infusion of FITC-coupled dextran (40 kDa, Sigma). For the in vitro cultured graft, the solution was infused directly into the vasculature via the arterial pedicle. The vascular perfusion was observed under a fluorescence microscope as described previously. FIG. 5 shows the retention of FITC-albumin in vessels formed following EPC perfusion of BioVacs.

### Low-Density Lipoprotein (LDL) Uptake

Endothelial cells in the reseeded vascular structures take up LDL, indicating endothelial function in vessels. Endothelial cells are able to incorporate LDL through receptor mediated endocytosis. In order to monitor the EC's metabolic function, the cells inside the vessel structure were exposed to 10 μg/ml AcLDL (Invitrogen) for 4 h at 37° C. by infusing the solution through the arterial pedicle of the vascularized scaffold. Nuclei were stained by incubation of 2 drops of NucBlue™ Live ReadyProbes™ (Invitrogen) per 1 ml assay solution for 30 min. A standard fluorescence microscope served for visualization. FIG. 6 shows seeded BioVac-LDL uptake into endothelial cells (red) in vessles generated by seeding decellularized vessels with EPCs.

### Graft Implantation

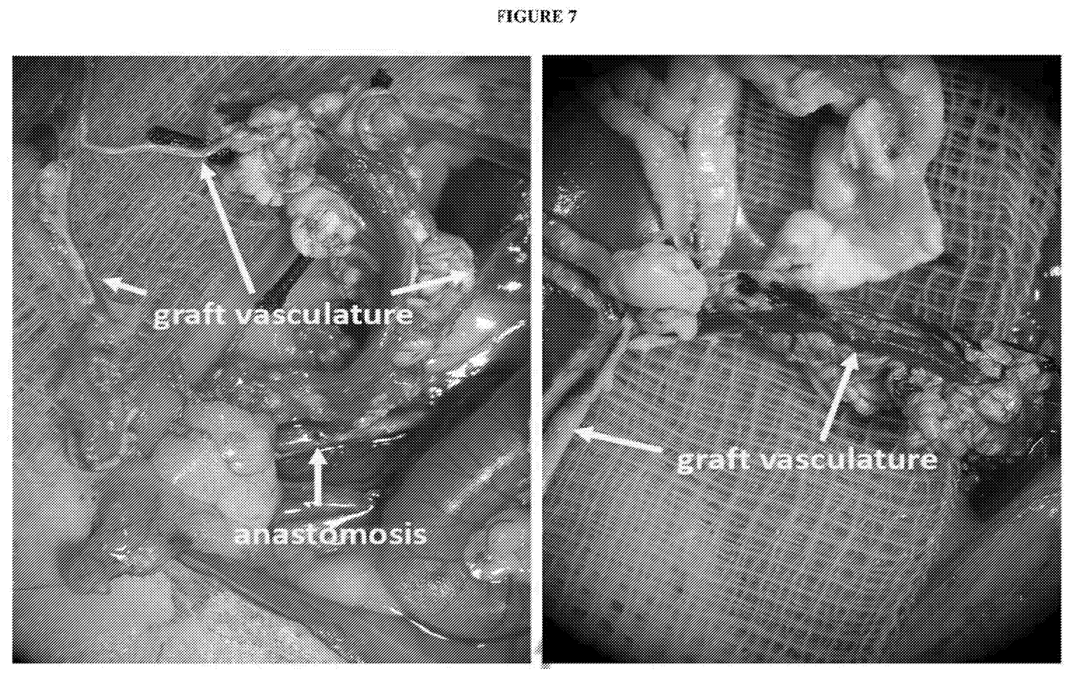

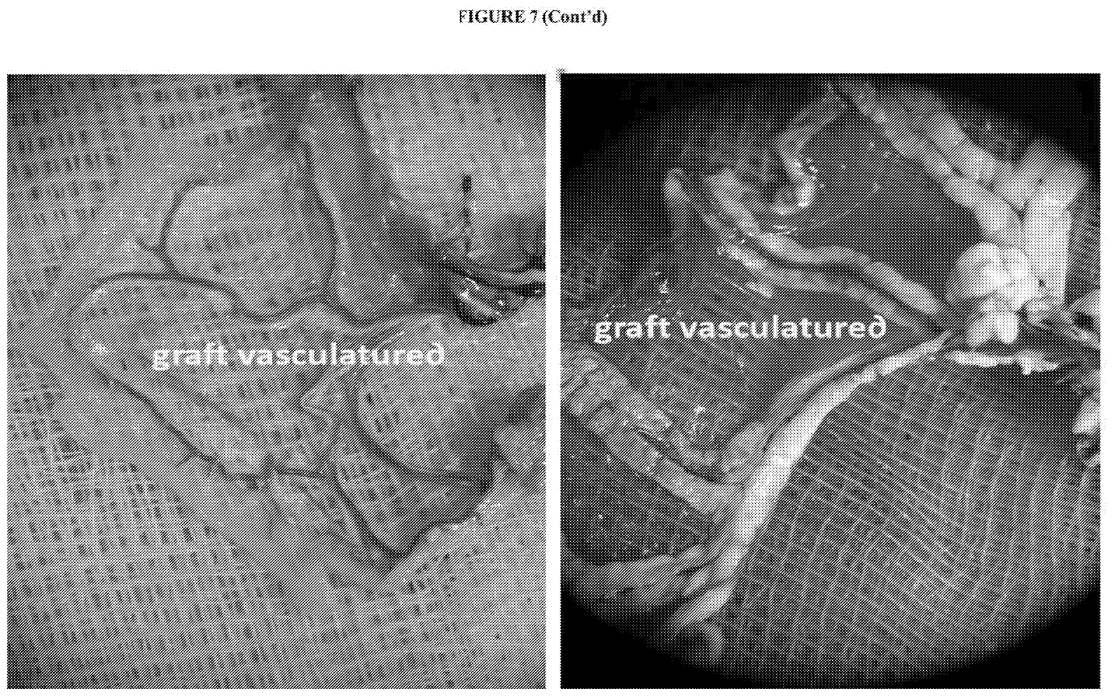

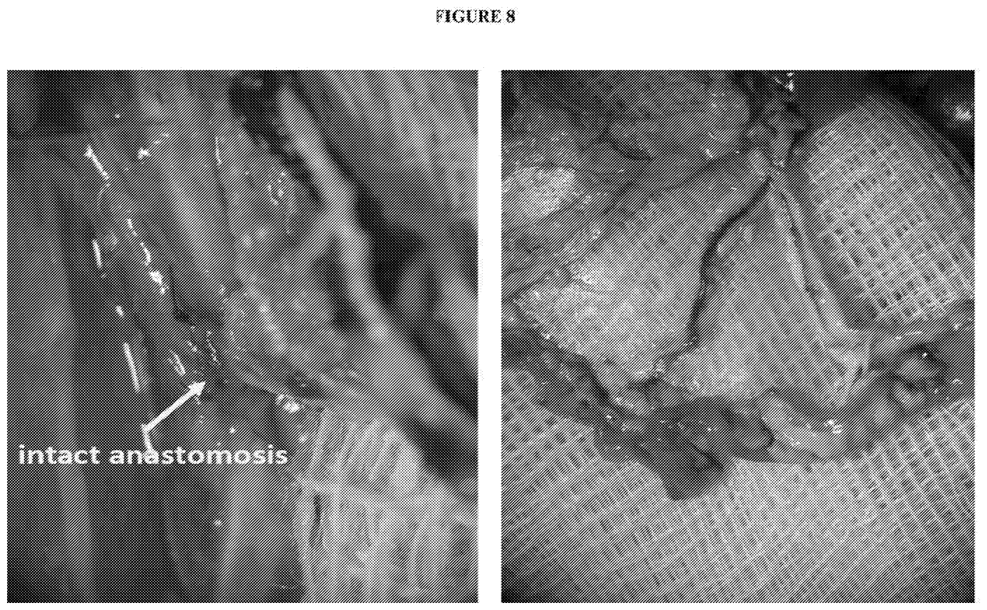

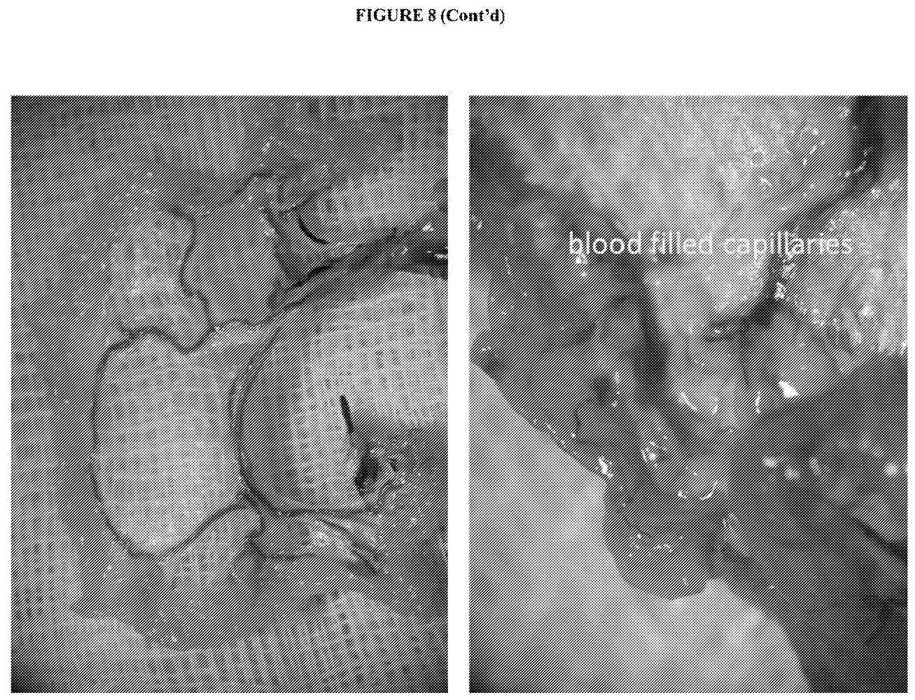

Reseeded vessels maintain their architecture and function in vivo and successfully serve to sustain blood flow. Graft implantation into the regio abdominalis of rats was conducted in female NIH Foxn1nu rats (age: 8 weeks) obtained from Charles River. General anesthesia was induced by continuous isoflurane inhalation-anesthesia with intraoperative analgesia (Carprofen, 5 mg/kg s.c.). The abdominal cavity was opened by median laparotomy. The infrarenal aorta abdominalis and the infrahepatic vena cava were dissected from fat and connective tissue. After clamping the vessels proximal and distal an incision of about 3 mm was made for the subsequent anastomosis. The mBioVaSc's artery was anastomosed side-to-end to the aorta abdominalis and the scaffolds vein to the vena cava. After examination of the patency of the anastomosis and the pervading of the scaffold with blood the abdominal cavity was closed occluding the abdominal musculature and closure of the skin with sutures. FIGS. 7 and 8 show the functional integrity of construct vasculature following transplantation for 3 days. The sites of anastomosis, blood-filled large vessels and capillaries of the graft vasculature are indicated.

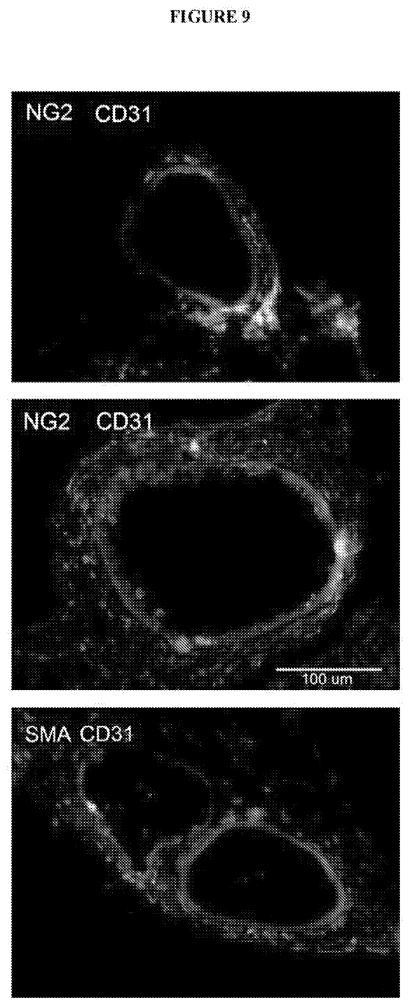

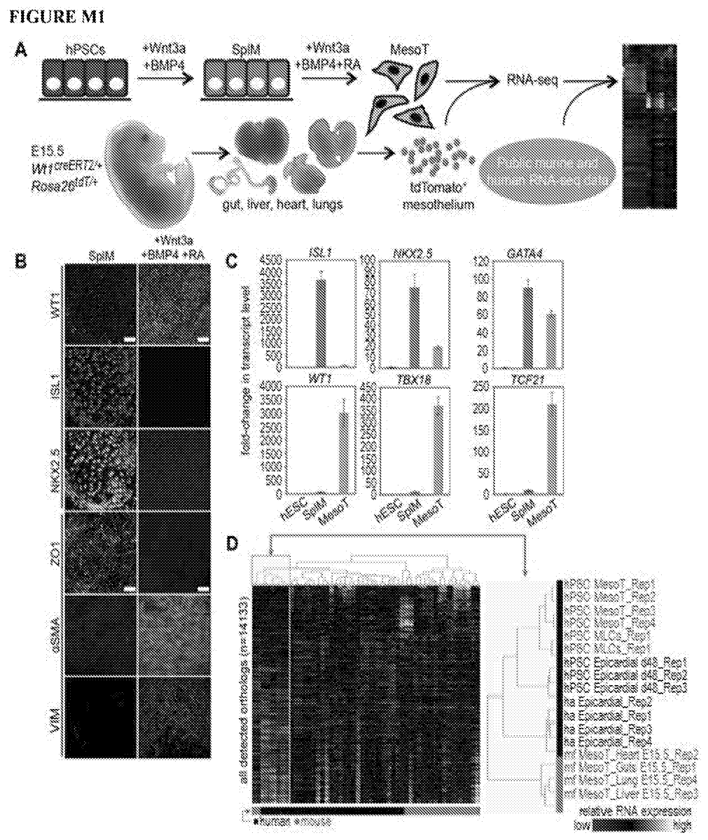

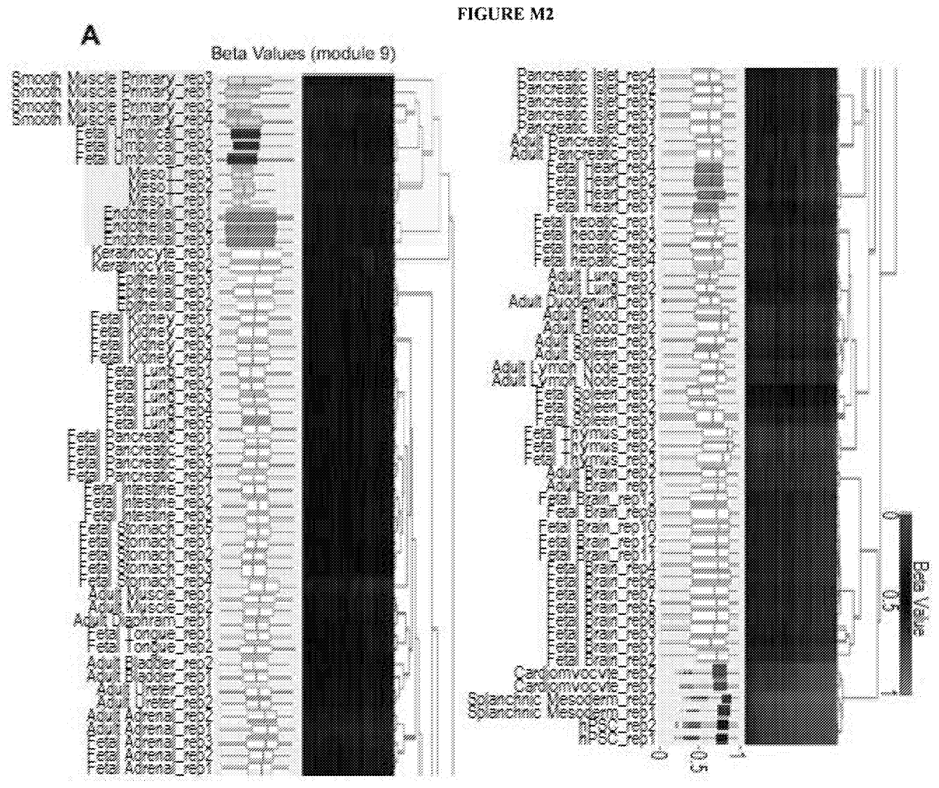

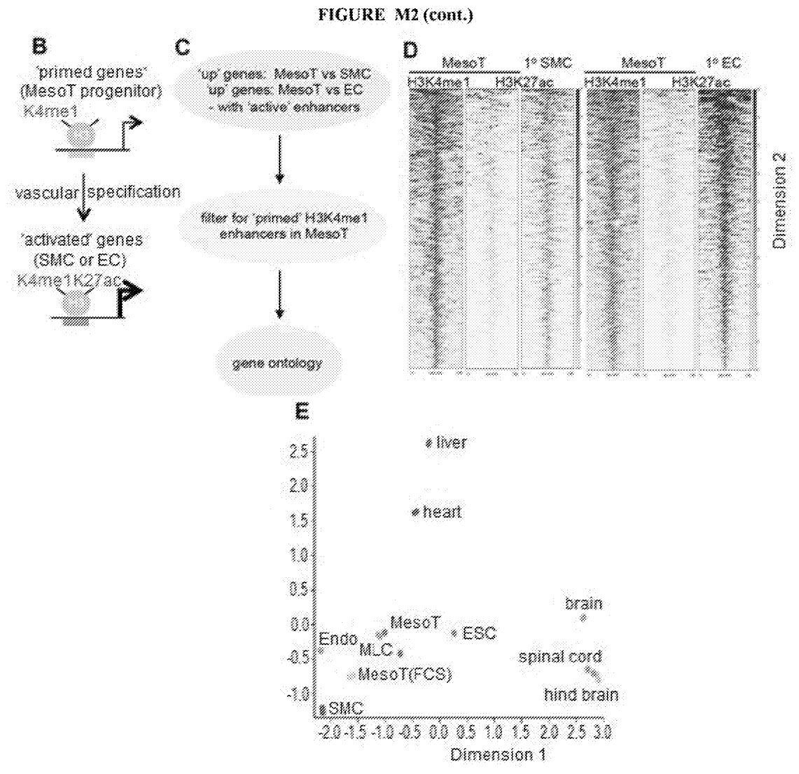

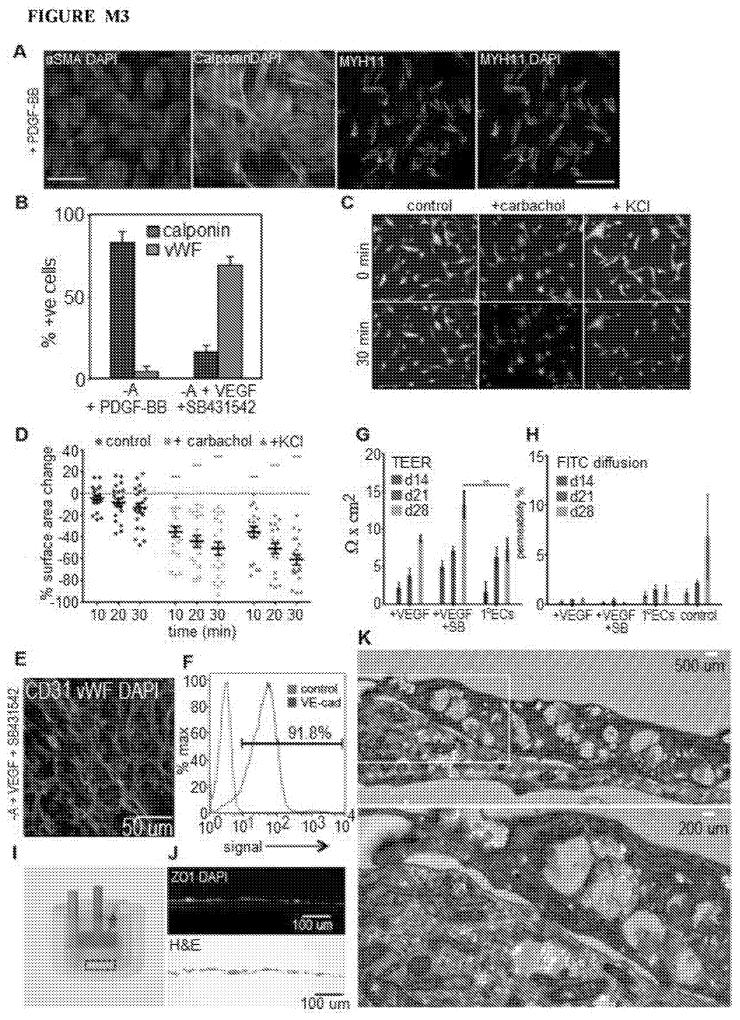

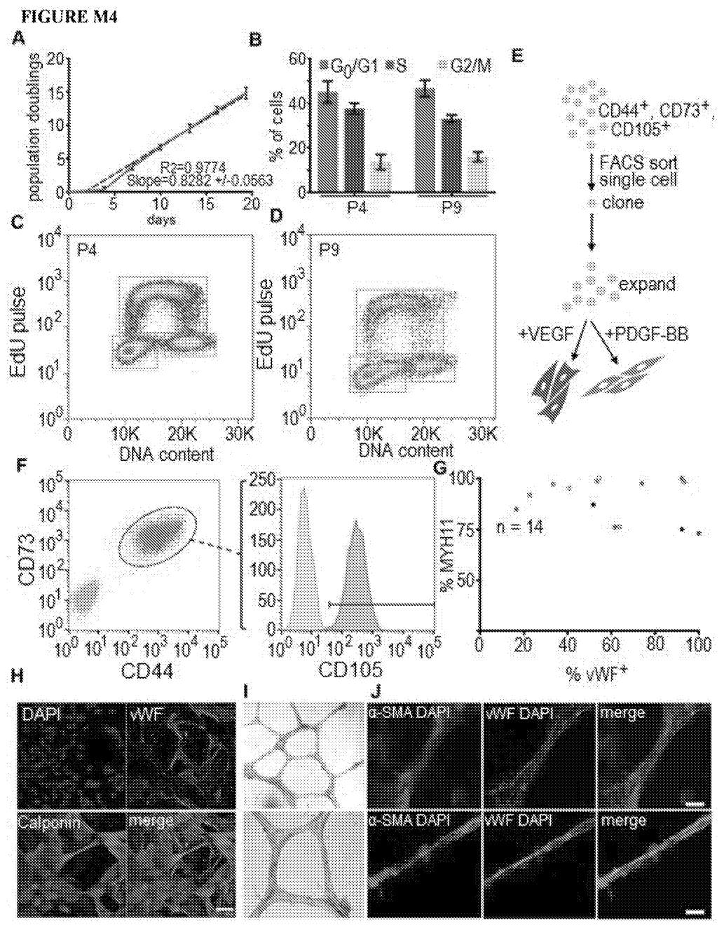

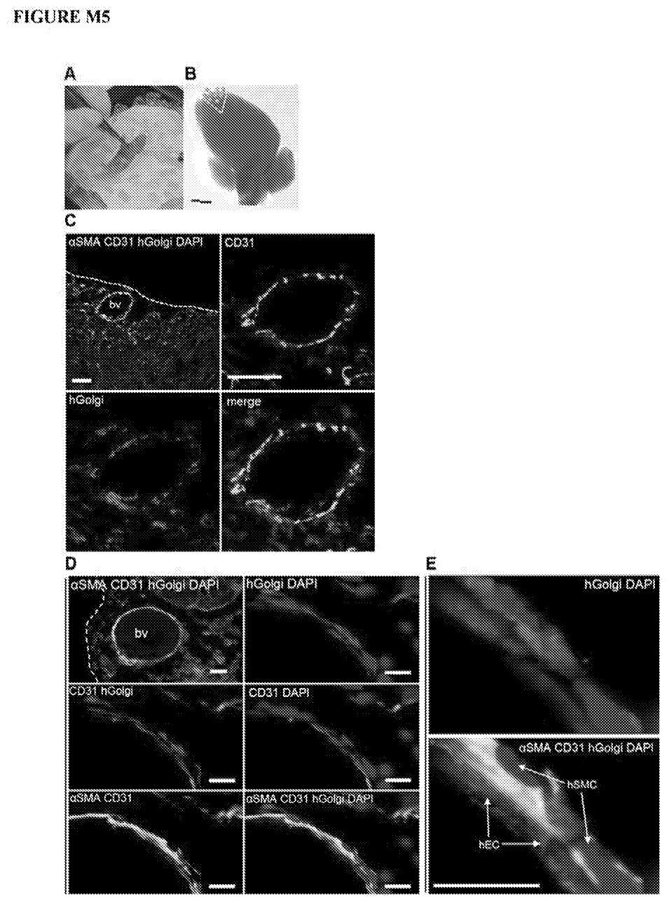

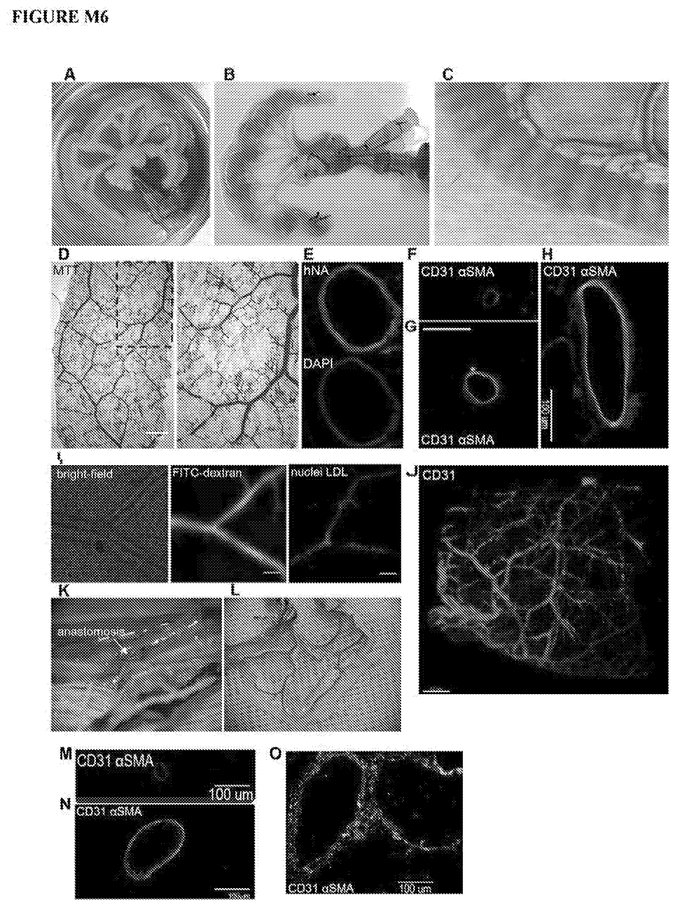

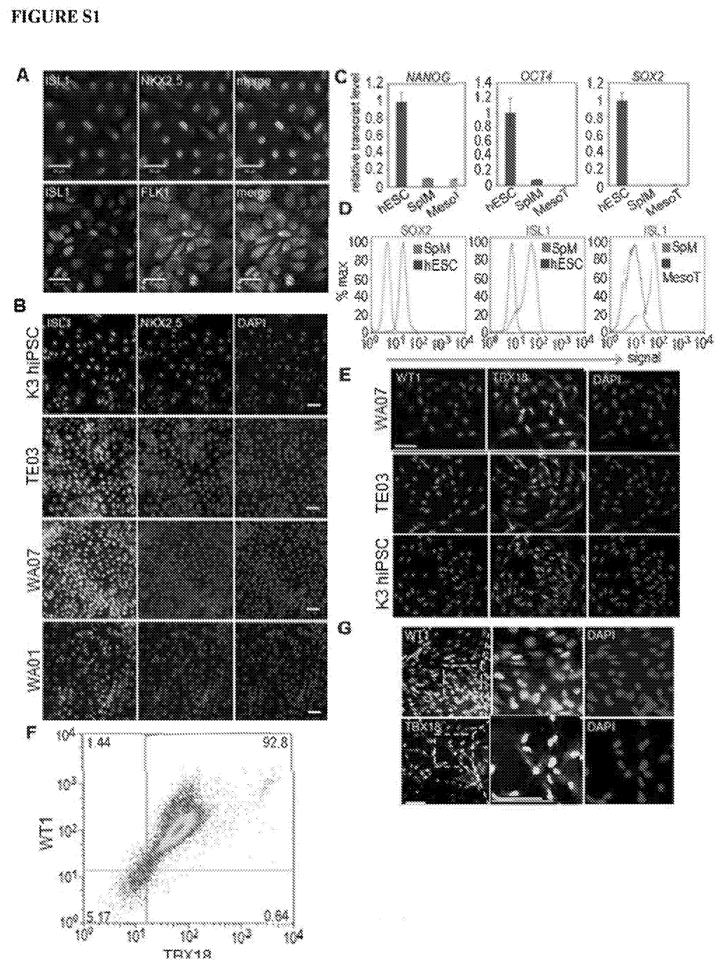

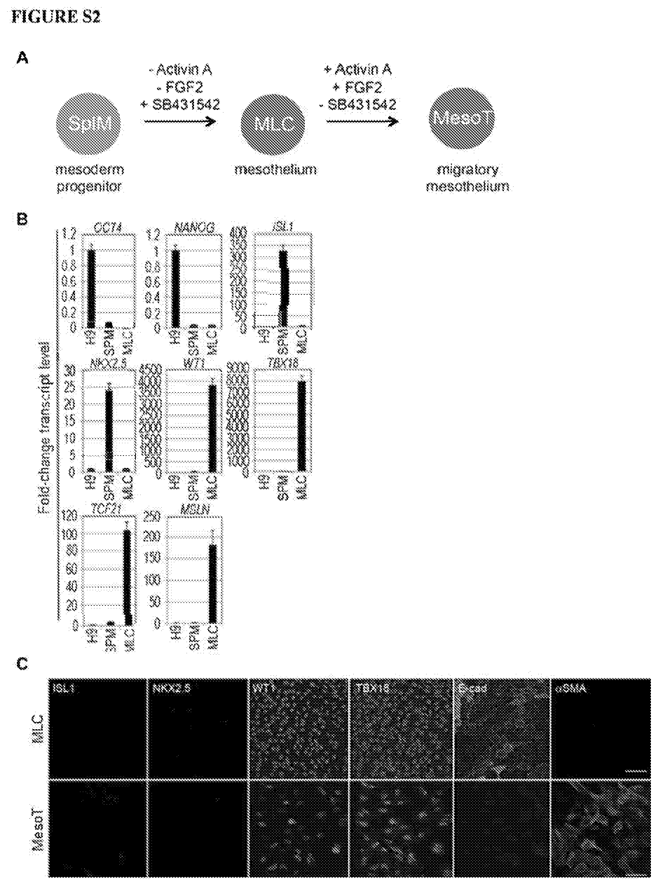

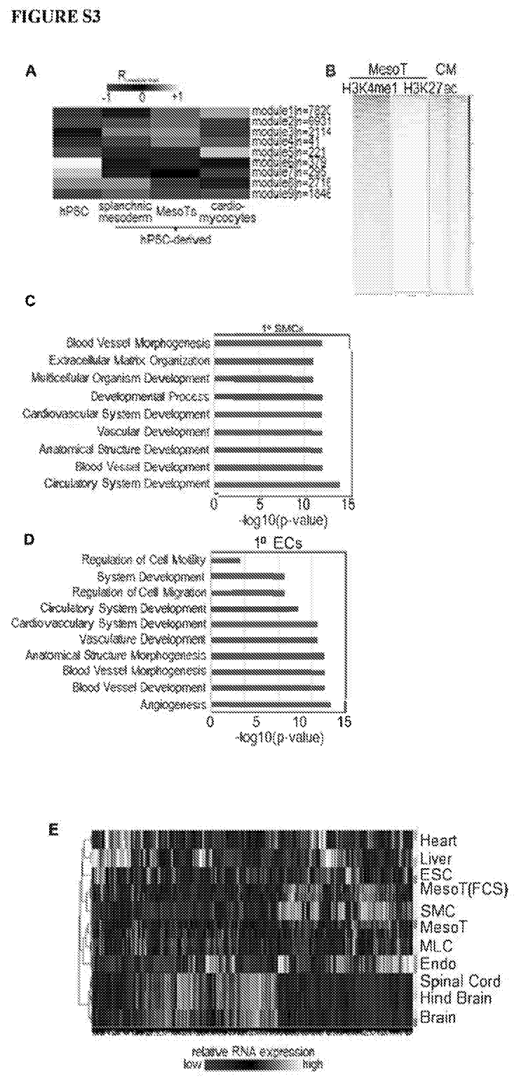

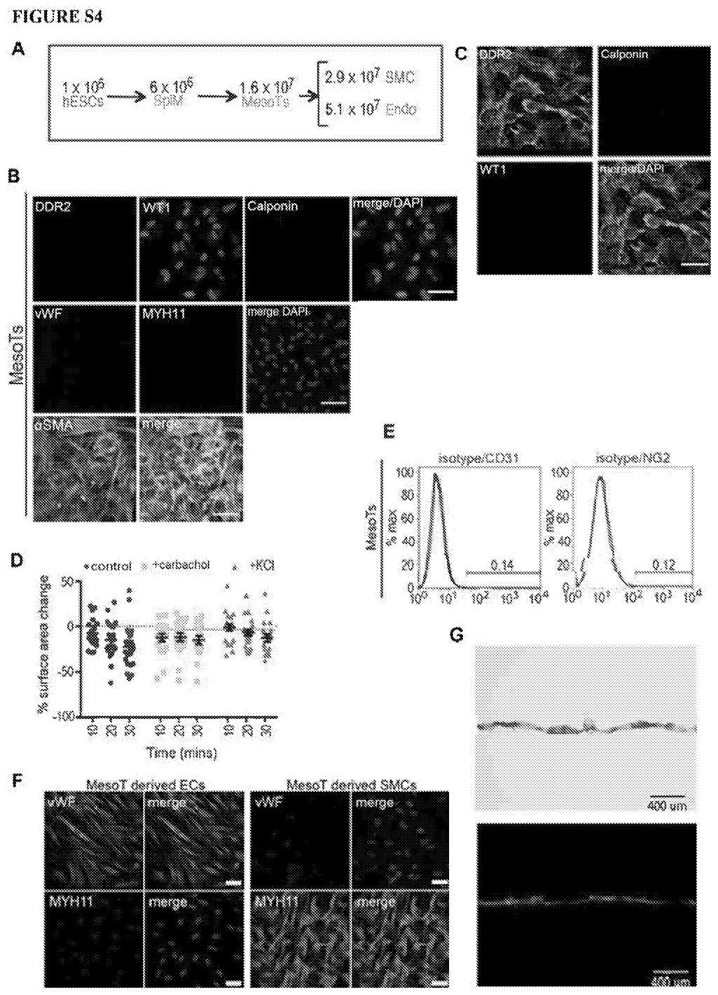

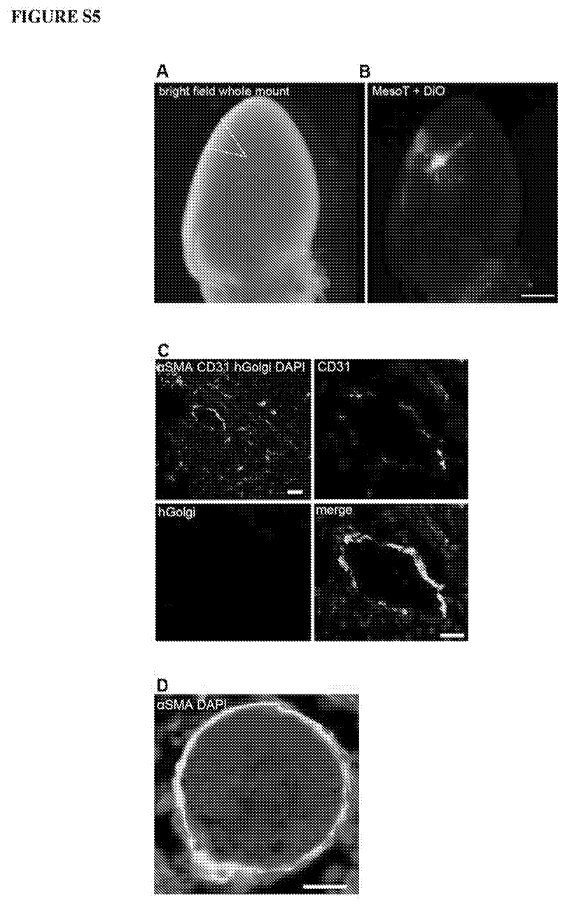

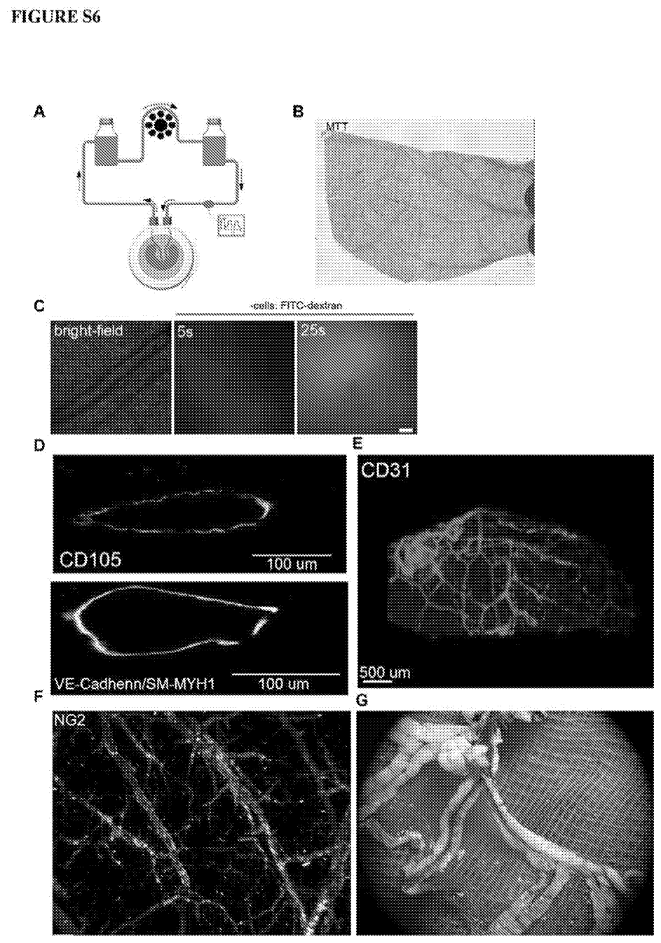

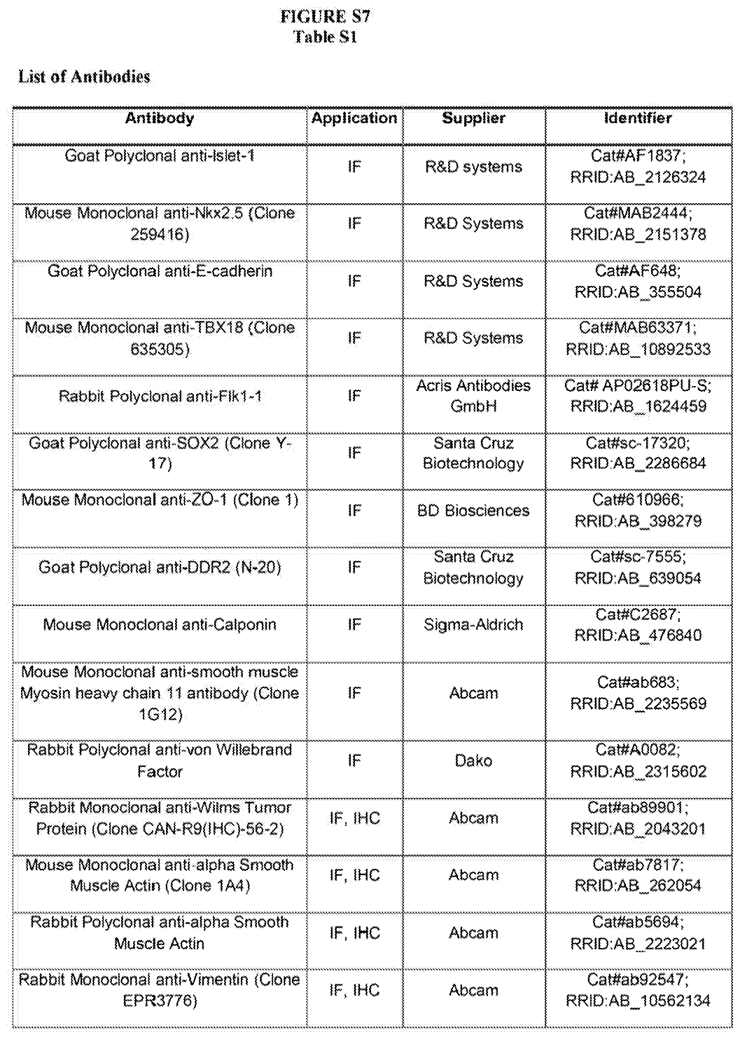

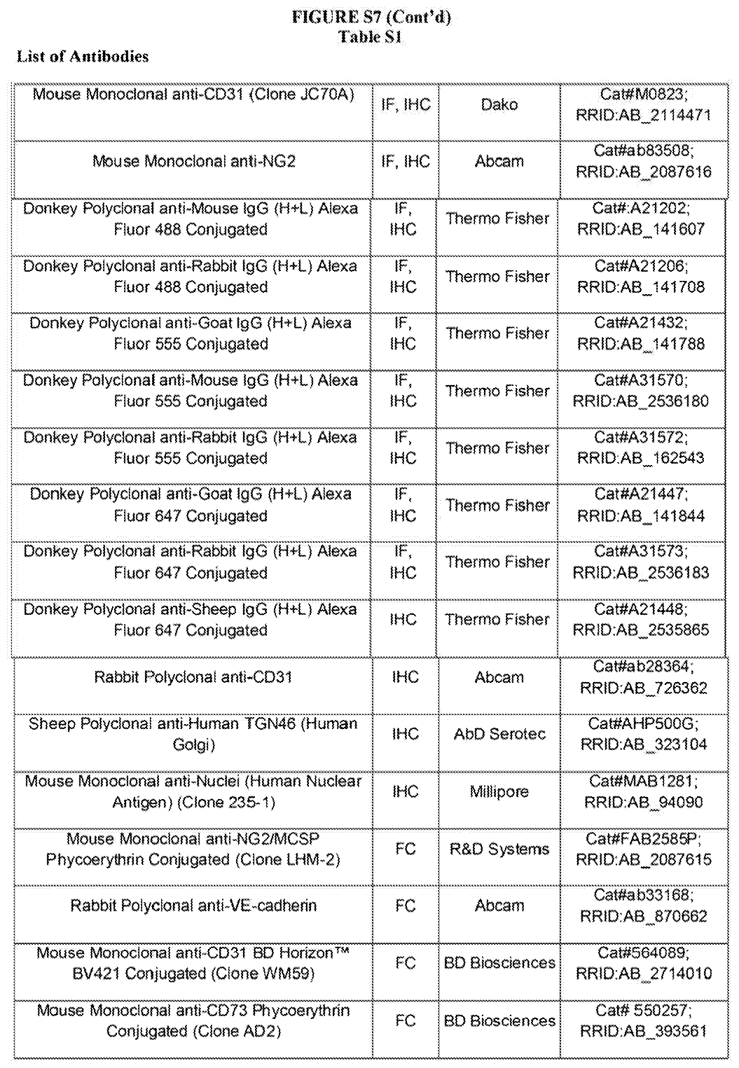

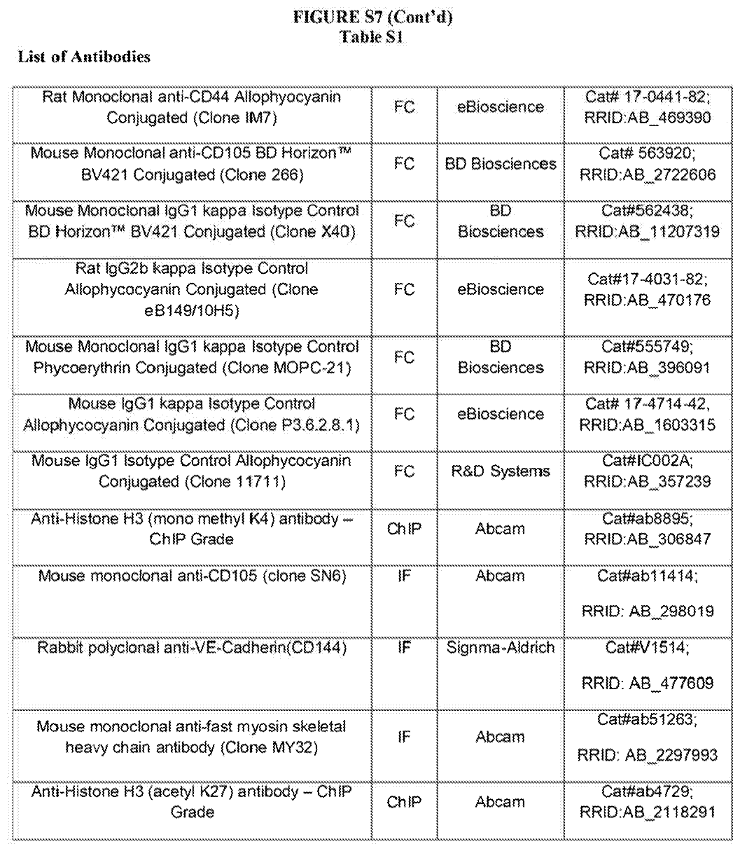

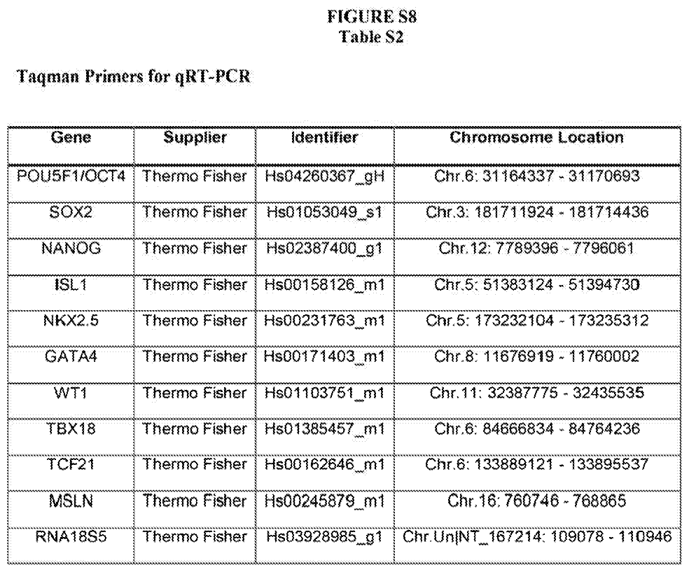

The vasculature of EPC-seeded constructs according to the present invention maintain their functional and structural integrity in vivo. FIG. 9 shows sections of EPC-seeded graft recovered 3 days after transplantation. Sections were probed with antibodies recognizing NG2, SMA and CD31 as indicated.

### Second Set of Experiments

In this second set of experiments, the inventor describes a novel, human pluripotent stem cell-derived vascular progenitor (EPC/MesoT) cell of the mesothelium lineage. EPC/MesoT cells are multipotent and generate smooth muscle cells, endothelial cells and pericytes and self-assemble into vessel-like networks in vitro). EPC/MesoT cells transplanted into mechanically damaged neonatal mouse heart migrate into the injured tissue and contribute to nascent coronary vessels in the repair zone. When seeded onto decellularized vascular scaffolds, EPC/MesoT cells differentiate into the major vascular lineages and self-assemble into vasculature capable of supporting peripheral blood flow following transplantation. These findings demonstrate in vivo functionality and the potential utility of EPC/MesoT cells in vascular engineering applications.

### Experimental Methods

### Cell Culture and Differentiations

Maintenance of pluripotent cells (Cliff et al., 2017) was as previously described. Briefly, WA09 (Sex: female, WiCell, NIHhESC-104062), WA07 (Sex: male, WiCell, NIHhESC-10-0061), WA01 (Sex: male, WiCell, NIHhESC-10-0043), TE03 (Sex: female, WiCell, NIHhESC-13-0204) human embryonic stem cells (hESCs) and K3 human induced pluripotent stem cells (hiPSCs), a gift from Stephen Duncan (Si-Tayeb et al., 2010), were maintained in chemically-defined media (CDM)(Wang et al., 2007), seeded at 5×10⁴ cells/cm² on Geltrex (Thermo Fisher, A1413302) coated plates at 1:200 dilution in DMEM/F12 w/o glutamine (Corning, 15-090-CM), and passaged with Accutase (Innovative Cell Technologies, AT104) upon confluency.

CDM consists of DMEM/F12 w/o glutamine supplemented with 1× nonessential amino acids (Corning, 25-025-CI), 1× antimycotic/antibiotic (Corning, 30-004-CI), 1× trace elements A (Corning, 25-021-CI), 1× trace elements B (Corning, 25-022-CI), 1× trace elements C (Corning, 25-023-CI), 2 mM L-alanyl-L-glutamine (Corning, 25-015-CI), 10 μg/ml transferrin (HOLO), human plasma, tissue culture grade (Athens Research and Technology, 16-16-032001-LEL), 2% Probumin® bovine serum albumin life science grade (EMD Millipore, 821005), 0.1 mM β-mercaptoethanol (Gibco, 21985023), 50 μg/mL ascorbic acid (Sigma-Aldrich, A8960), 10 ng/ml rhHeregulin β-1 (Peprotech, AF-100-03), 200 ng/ml LONG® R3 IGF-I human (Sigma-Aldrich, 85580C), 10 ng/ml rhActivin A (R&D Systems, 338-AC) and 8 ng/ml rhFGF basic (R&D Systems, 4114-TC).

To generate splanchnic mesoderm (SplM) (Berger et al., 2016), hPSCs were passaged as above and reseeded at 5×10⁴ cells/cm² (WA09) or 1×10⁵ cells/cm² (WA01, WA07, TE03 and K3) onto Geltrex coated plates into CDM supplemented with 25 ng/ml rhWNT3a (R&D Systems, 5036-WN) and 100 ng/ml rhBMP4 (R&D Systems, 314-BP) for 4 days with media changed daily.

Mesothelium-like cells (MLCs) were generated by passaging SplM with TrypLE select (Thermo Fisher, A1217701) diluted to 1× in a salt balanced solution consisting of PBS (Corning, 21-031), sodium chloride (1.85 g/L, Fisher Scientific, S640), and 0.5 M EDTA (1 ml/L. Thermo Fisher, AM9260G). Cells were reseeded at 1.5×10⁵ cells/cm² in CDM without FGF2 or Activin A, and with 25 ng/ml rhWNT3a, 50 ng/ml BMP4, 20 μM SB431542 (Tocris, 1614), 4 μM all-trans retinoic acid (Sigma-Aldrich, R2625) for 18 days with media changed daily.

Migratory mesothelium (MesoT) is generated from SplM and does not require passaging and reseeding of cells. On day 4 of SplM induction, CDM is merely supplemented with 25 ng/ml rhWNT3a, 50 ng/ml rhBMP4 and 4 μM all-trans retinoic acid for an additional 14-18 days. To generate EPC/MesoT cells from MLCs, cells are passaged with Tryp LE select and reseeded at 1.5×10⁵ cells/cm² in CDM supplemented with 25 ng/ml rhWNT3a, 50 ng/ml rhBMP4 and 4 μM all-trans retinoic acid for an additional 18 days. To self-renew MesoT (FBS), cells were passaged with collagenase type IV (800 units/ml, Thermo Fisher, 17104-019) followed by 1× TrypLE Select and cultured with 10% fetal bovine serum (Atlanta Biologicals, S10250), Minimal Essential Media, Alpha (Corning, 15-012-CV), lx antibiotic/antimycotic, 2 mM L-alanyl-L-glutamine and 8 ng/ml FGF2 at an initial seeding density of 5×10⁴/cm² on plastic polystyrene plates then 1×10⁴/cm² for subsequent passages using 1×TrypLE Select. Media was changed daily.

Downstream lineages were generated by passaging MesoTs/EPCs as above and reseeding at a density of 1.5×10⁵/cm² on Geltrex coated plates. Activin A was removed from CDM and supplemented with the following: 50 ng/ml rhVEGF-A₁₆₅ (R&D Systems, 293-VE) and 20 μM SB431542 for endothelial cell differentiations; 50 ng/m PDGF-BB (R&D Systems, 220-BB) for smooth muscle cell (SMC) differentiations, and 50 ng/ml PDGF-AA (R&D Systems, 221-AA) for fibroblasts. Each differentiation was allowed to proceed for 12 days with media changed every other day. Removal of SB431542 from endothelial cell media was used to generate mixtures of endothelial cells and smooth muscle cells that can self-assemble in vitro into tube structures.

### Immunofluorescence Analysis of Fixed Cells

Immunofluorescence analysis was performed on 4% paraformaldehyde (VWR, 15170) fixed cells (10 min) in the presence of 10% donkey serum (Equitech-Bio, SD30) and 0.25% Triton X-100 (Fisher Scientific, BP151) followed by visualization using 2.5% donkey serum in PBS with fluorescent conjugated secondary antibodies. Primary antibodies were incubated overnight at 4° C. followed by conjugated secondary antibodies for 1 hour at room temperature (RT) in the dark. Nuclei were counterstained with 4′,6-Diamidino-2-phenylindole dihydrochloride (DAPI Sigma-Aldrich, D9542) for 5 min and cover slips were affixed with ProLong Diamond Antifade (Thermo Fisher, P36961). Fluorescent cells were visualized on a Leica DM6000 B and BioTek Lionheart FX. Confocal images were obtained on an Olympus FV1200 laser scanning confocal microscope. All antibodies are listed in FIG. S7, Table S1.

qRT-PCR

mRNA was isolated using the E.Z.N.A.® Total RNA Kit I (Omega Bio-Tek, R6834) followed by quantification using a Biotek Synergy 2 plate reader. cDNA was made using iSCRIPT cDNA kit (Bio-Rad, 1708841). qRT-PCR was performed using TaqMan Universal PCR Master Mix No AmpErase UNG (Thermo Fisher, 4324020) and TaqMan Primers on a ViiA7 Real-Time PCR System (Life Technologies). TaqMan probe mixes are listed in FIG. S8, Table S2.

### Flow Cytometry, Cell Cycle Analysis and Clonal Analysis

Cells were collected as single cell suspensions following removal from the culture as noted above then analyzed by flow cytometry using a CyAn ADP (Beckman Coulter, Hialeah, Fla.). Cells were stained with specific flow antibodies per manufacturer recommendations with isotype controls. Intracellular markers were analyzed using Flow Cytometry Permeabilization/Wash Buffer I (1×, R&D Systems, FC009). Cell cycle analysis of MesoT cells was performed using a Click-iT™ Plus EdU Alexa Fluor™ 647 Flow Cytometry Assay Kit (Invitrogen, C10634) with 1-hour incubation. Population doublings were calculated using the following equation (Takeuchi et al., 2009):

$$\frac{log \frac{End Cell Number}{Starting Cell Number}}{log 2}$$

For clonal analysis, CD44⁺/CD73⁺/CD105⁺ MesoT cells were single cell sorted onto 96 well plates (VWR) using 50/50 fresh and preconditioned media with a MoFlo XDP sorter (Beckman Coulter, Hialeah, Fla.). After amplification, cells were passaged to downstream lineages using 2% fetal bovine serum, Minimal Essential Media, Alpha, 1× antibiotic/antimycotic, 2 mM L-alanyl-L-glutamine and 50 ng/m rhVEGF-A₁₆₅ (endothelium) or 50 ng/ml PDGF-BB (smooth muscle). Multipotency was assessed by confocal imaging on an Olympus FV1200 confocal microscope and quantified with ImageJ software (NIH website: imagj.nih.gov/ij/). All flow and sorting analysis used FlowJo® cell analysis software (FlowJo, LLC, Ashland, Oreg.).

### Isolation of Embryonic Mouse Mesothelium

Wt^{creERT2/+} Rosa26^{tdT/+} embryonic mice were generated by crossing Wt1^{creERT2/+} mice (Zhou et al., 2008) with a knock-in of tamoxifen inducible cre-recombinase in the Wt1 locus (Jackson Laboratory, 010912) and Rosa26^{tdT/tdT} (Jackson Laboratory, 007914) mice with a loxP-flanked STOP cassette preventing transcription of a CAG promoter-driven red fluorescent protein variant, tdTomato (Madisen et al., 2010). At E12.5, pregnant dams were injected with 300 μl of 10 mg/ml tamoxifen (Sigma-Aldrich, T5648) in corn oil solution (Sigma-Aldrich, 8267) intra-peritoneally at 24-hour intervals for three consecutive days. Embryos were harvested at E15.5 and the heart, lung, liver and gut were carefully isolated using sterile technique. The outer mesothelial layer was digested by exposing intact organs to a dissociation buffer containing 1 mg/ml collagenase IV and 0.05% trypsin-EDTA (Thermo Fisher, 25300054) as described previously (Zhou and Pu, 2012). Briefly, the intact organs were repeatedly digested (7-8 times) in collagenase IV-trypsin dissociation buffer at 6-7 min intervals on a 37° C. shaker and the supernatants from each digest neutralized with horse serum (Atlanta Biologicals, S12150), pooled and filtered through a 70 μM nylon mesh. Cells isolated from nine embryos were pooled for analysis in order to obtain sufficient material. Digested samples were suspended in FACS buffer (0.2% Probumin® in PBS). tdTomato cell fractions were sorted using a MoFlo XDP and RNA was extracted for differential gene expression analysis.

### RNA-Seq Analysis

RNA from tdTomato⁺ fractions along with RNA from hESC-derived MesoT and MLCs were extracted with Trizol (Invitrogen, 15596-026) and purified with RNeasy mini kit (Qiagen, 74104) according to the manufacturer's instructions. RNA yield was determined using the NanoDrop ND-1000 spectrophotometer (NanoDrop Technologies) and purified RNA (1 μg) from each sample was submitted to the Hudson Alpha Institute (Huntsville, Ala.) for polyA⁺ RNA sequencing (HiSeq v4 50 PE, 25 million reads per sample) and deposited under GSE113090. Additional raw data for human adult and fetal tissues were downloaded from the Sequence Read Archive (ERP003613, SRP001371) while additional mouse adult and fetal tissues were downloaded from ENCODE and the Sequence Read Archive (SRP049248, SRP018511). When necessary,*.sra formatted data were converted to the *. fastq format using the NCBI SRA Toolkit (version 2.4.1). Data quality was assessed using FastQC (version 0.10.1) before being mapped to known Ensemble genes (GRCh37/NCBIM37) using Tophat2 (version 2.0.13) (Kim et al., 2013). Raw read counts for each gene were obtained using Subread (version 1.4.2)(Liao et al., 2013). Manipulation of raw sequence data was executed on an 8-core UNIX node with 48G RAM.

Raw mouse and human read count data were read into R (R Core Team, 2013) and combined into species specific data frames for differential expression analyses using linear modeling strategies. The mouse sample set was comprised of four FACS-sorted mesothelium samples and replicated samples from 18 mouse tissues (40 total samples). The human sample set was comprised of 2 replicates of hPSC-derived MLCs, 4 replicates of hPSC-derived MesoT, and replicated samples from 25 human tissues (62 total samples). The RNA-seq expression sets were filtered to include genes having at least 5 reads in 2 or more samples (nₘₒᵤₛₑ=18836, nₕᵤₘₐₙ=21761). Raw read counts were normalized using the trimmed mean of M-values (TMM) method (Robinson and Oshlack, 2010) and precision weights were calculated using voom (Law et al., 2014) prior to differential analysis using the Limma empirical Bayes analysis pipeline (Smyth, 2004).

### Principal Component Analysis

For principal component analysis, each sample has two biological replicates. EPC/MesoT (CDM & FCS) and mesothelium (MLC) were deposited under GSE113090. WA09 hESCs, human coronary artery endothelium, human coronary artery smooth muscle cells, fetal liver, heart and brain, fetal hindbrain and spinal cord datasets were obtained from GSE10165S, PRJDB4498, GSE51878, GSE63634, GSM2229922. Raw data were aligned to human reference genome (hg19) using HIAST2 (version 2.1.0) with default parameters. Aligned reads were sorted with Samtools (version 1.3.1) (Li et al., 2009) and the number of reads mapping to each gene were counted by HTseq (version 0.6.1p1). All counting files from HTseq were imported into R studio (version 1.1.423)(RStudio Team, 2015). In order to generate the PCA plot, data were pre-processed using Limma edgeR (McCarthy et al., 2012; Robinson et al., 2010) to normalize sequencing depth and gene expression distributions as well as to remove low counts. The PCA plot was made by Glimma (Su et al., 2017) using the top 50% of genes.

### DNA Methylation Analysis

Bisulfite conversion was performed using the EZ DNA Methylation Kit (Zymo Research, D500) according to the manufacturer's instructions, Whole genome amplification, fragmentation and preparation of the DNA for hybridization were performed using the Infinium HumanMethylation450 BeadChip kit (Illumina, WG-314-1001) as described in the manufacturer's protocol. Illumina IDATS were read directly into R and normalized using Subset-quantile within array normalization (Maksimovic et al., 2012). A summary of all DNA methylation samples analyzed in this study were deposited under GSE116754. Public datasets were downloaded from GSE31848. Dynamic methylation analyses were performed using Limma in R (Ritchie et al., 2015). Briefly, triplicate EPC/MesoT samples were fit to a linear model along with undifferentiated hPSC, hPSC-derived SpiM and hPSC-derived cardiomyocytes (all biological duplicates). Dynamic methylation loci were identified by selecting probes with FDR<=0.01 in a targeted t-test comparing EPC/MesoT to undifferentiated hPSCs. These loci were further filtered by removing probes where the maximum difference in Beta values was <0.3 between any of the sample group averages used to fit the linear model (n=22561). DNA methylation modules were identified using weighted gene correlation network analysis in R (WGCNA) (Langfelder and Horvath, 2008). Filtered sets of significant differentially methylated loci and methylation module assignments were provided.

### ChIP-Seq and Gene Ontology Analysis

ChIP-seq was performed as previously described (Singh et al., 2015). Briefly, EPC/MesoT cells were chemically crosslinked with 1% formaldehyde (Thermo Scientific, 28906) for 10 min at room temperature, followed by quenching with 2.5 M glycine (Sigma-Aldrich, G8898) for 5 min. Samples were rinsed with cold 1×PBS once, harvested by centrifugation and stored at −80° C. before use. Cross linked DNA was fragmented by sonication on a Covaris S220 with 140V peak power, a duty factor of 5 and 200 cycles for 10 min. H3K4 mono-methylation (Abcam, ab8895) and H3K27 acetylation (Abcam, ab4729) antibodies were incubated with 50 μl Protein G Dynabeads (Invitrogen, 10004D) on a shaker at 4° C. for 2 hours. Fragmented DNA, antibodies and beads were then incubated together on a shaker at 4° C. overnight, approximately 5 million cells per reaction. Cross-linking was reversed by adding 20 mg/ml Proteinase K (Qiagen, 19131) at 55° C. for 30-40 min followed by incubation at 65° C. for 8 hours. The final library was submitted to Hudson Alpha Institute for 50 bp single-end sequencing. For each sample, ˜25 M reads were obtained, and input samples were used as controls. EPC/MesoT H3K4me1 and H3K27ac ChIP-seq data was deposited under GEO accession GSE113090.

All ChIP-seq raw data were aligned to UCSC human reference genome (hg19) using Bowtie2 software (version2.2.9)(Langmead et al., 2009) and only uniquely aligned reads were retained. Samtools was used to sort aligned reads into genomic order for further analysis. For visualization, sorted BAM files were indexed with Samtools and genome coverage tracks (bigwig files) were generated using Deeptools (version 2.3.1) (Ramirez et al., 2016) with parameters of —binsize 10, —extendReads 200. Normalization was performed by reads per genome content (RPGC). H3K4me1 peaks, H3K27ac peaks and differentially regulated H3K27ac regions between EPC/MesoT, human coronary artery smooth muscle cells (HCASMC) and human umbilical vein endothelial cells (HUVEC) and were detected using MACS2 (version 2.1.1) by calling broad peaks and differential binding events. All reads were extended from 5′ to 3′ direction to a final length of 200 bp. The enriched H3K4me1 and H3K27ac peaks were defined as the regions with significant enrichment of FDR<0.01 relative to respective input control reads. Differentially regulated H3K27ac sites were obtained with a cutoff of logo likelihood ratio between two samples. The overlapping regions of H3K4me1 and H3K27ac sites and peaks annotation were analyzed by ChIPseek (Chen et al., 2014b). Potential primed enhancers were defined as sites which have highly enriched H3K27ac signals in HCASMC and HUVEC compared to EPC/MesoT and were also marked with H3K4me1 in EPC/MesoT. A list of lineage specific genes with primed enhancers regions in EPC/MesoT were obtained in R by intersecting HCASMC- and HUVEC-specific genes (identified as fold change>4, compared to EPC/MesoT, from RNA seq analysis) with annotation tables from potential primed enhancers. The H3K4me1 and H3K27ac heat maps were generated with Deeptools. The matrix was built on a list of genes having primed enhancer along with their corresponding H3K4me1 peak regions and signal files of EPC/MesoT H3K4me1 bigwig file and HCASMC and HUVEC H3K27ac bigwig files with bin size of 50 bp. The signals were restricted to 5 kb around the central region of H3K4me1 peaks. The Gene Ontology enrichment analysis was performed on HCASMC- and HUVEC-specific genes with primed enhancers in MesoTs with Panther (Mi et al., 2017). The GEO accession numbers for HCASMC and HUVEC H3K27ac ChP-seq are GSM1876036 and GSM1009635, respectively.

### Contraction, Tube Formation, Transwell Barrier Assays

Day 12 SMCs differentiated from MesoTs/EPCs were labeled with DiO (Thermo Fisher, V22886) per manufacturer's instructions then replated at 3×10⁵ cells per cm² on Geltrex coated 12 well plates (Corning) for 24 hours. Cells were treated with 100 μM carbachol (Iyer et al., 2015)(Sigma-Aldrich, 212385), 50 mM KCl (Motherwell et al., 2017) (Fisher Scientific, P333-500) or left untreated (control) and monitored for 30 min using a BioTek Lionheart FX. Surface area change for 20 selected cells was measured with ImageJ. For tube formation assays, MesoT cells were cultured on Matrigel (VWR, 47743-715) in CDM (-Activin A) supplemented with 50 ng/ml rhVEGF-A₁₆₅ at a density of 1.5×10⁵ cells/cm². Media was changed every other day. Tubes were fixed and probed with antibodies as above.

To conduct the endothelial barrier assay, ThinCert™ Cell Culture Inserts (pore size 0.4 μm, Greiner Bio-One International, 662641) were coated with collagen IV (Sigma-Aldrich, C5533) and fibronectin (Thermo Fisher, 33016015). MesoT cells were seeded onto the PET membrane and cultured at 37° C. and 5% CO₂ using media to generate ECs or EC/SMC mixtures, as noted above, for 28 days under static conditions in a standard well-plate. Primary dermal microvascular endothelium, a gift from Heike Walles (Kress et al., 2018), cultured in VascuLifet® VEGF-Mv Endothelial Complete Kit (Lifeline Cell Technology, LL-005) were used as a control. Barrier integrity of cells was examined by trans-endothelial electrical resistance (TEER) measurements and a FITC-dextran (40 kDa, Sigma-Aldrich, FD40S) permeability assay. TEER values of the barrier separating the apical and basolateral compartment were determined using a hand-electrode (Millicell ERS-2, Millipore). Permeability of FITC-dextran was measured after application into the apical compartment on an orbital shaker (77 rpm) for 30 min. Cell junctions were visualized by osmium tetroxide (Sigma-Aldrich, 75633) contrasting followed by imaging on a JEM-2100 transmission electron microscope (JEOL, Tokyo, Japan).

### Transplantation of EPC/MesoT Cells in a Neonatal Mechanical Injury Model

Wt1^{creERT2/+}; Rosa26^{tdT/+} and Wt1^{+/+}; Rosa26^{tdT/+} neonatal mice were generated by crossing Wt1^{creERT2/+} and Rosa26^{tdT/tdT} mice and given an intragastric injection of tamoxifen (1 μg) dissolved in corn oil on day of birth (D0.5). Six hours after injection, neonatal mice were separated from their mothers and placed on ice for 5 min to induce hypothermia-based anesthesia (Wixson and Smiler, 1997). Hearts of neonatal mice were mechanically injured as previously described (Porrello et al., 2011). Briefly, a lateral thoracotomy in the 4^{th} intercostal space was performed to expose the apex of the heart. In the sham operated mice, after the heart was exposed, the heart was gently placed back into the chest cavity and the rib cage and chest wall were surgically sewn up using 6-0 non-absorbable Prolene sutures. In mechanically injured mice, the heart was resected using iridectomy scissors. In mice receiving treatment with MesoT, 1 million cells labeled with DiO cell labeling solution were suspended in 2 μl of Ca²⁺ and

Mg²⁺-free PBS and injected into the pericardial cavity before sewing up the rib cage and chest wall. Once surgery was complete, neonatal pups were allowed to recover by rapid warming and then returned to their mother. Hearts from each treatment condition were harvested at various times up to 30 days post-injury. Following removal, collected hearts were washed in PBS, fixed in 4% paraformaldehyde for 2 hours then cryo-protected overnight in 30% sucrose solution (Sigma-Aldrich, S0389)(both at 4° C.) and embedded in OCT (Fisher Scientific, 14-373-65). To assess the human cellular contribution to the injury site, cryo-sections of 10 μm thickness were probed with antibodies for human-specific Golgi (TGN46, AbD Serotec, AHP500G) or human nuclear antigen (Millipore, MAB1281) and cell lineage markers.
Recellularization of Vascular Scaffolds with MesoTs/EPCs

Jejunal scaffolds were isolated as described previously (Kress et al., 2018). Briefly, 6-8 week old anesthetized Lewis rats were subject to a median laparotomy to isolate a jejenal segment containing its arterial and venous pedicle. The animal was systemically perfused with heparin (100 IE/kg) with the draining vein and feeding artery cannulated with a 20-Guage catheter and flushed with phosphate-buffered saline. Decellularization of the jejenum was performed by perfusion with deionized water at 4° C. for 24 hrs followed by perfusion with 4% sodium deoxycholate at room temperature for 4 hrs and then 1 mg/ml DNase I for a further 3 hrs at room temperature. Each step was followed by a PBS wash.

MesoTs/EPCs were injected through the arterial and venous cannulas into the vascular tree of a decellularized rat jejenum. Optimization of recellularization was achieved by injecting a bolus of cells on three consecutive days. On day 1, 0.5-1×10⁶ cells in 0.5 ml cell culture medium were injected per cannula with an infusion rate of 4 ml/min. Following a one-hour static incubation, allowing for cellular adherence, the injection and static incubation were repeated. To promote functional maturation of the cellular layer on the vascular bed, the scaffold was connected to a bioreactor system to mimic physiological blood flow conditions by perfusion culture (Kress et al., 2018). Cell injection and static adherence were repeated once per arterial and venous cannula per day with subsequent perfusion culture overnight for three consecutive days. After the third day of injection, media was perfused for up to 28 days in the bioreactor. Temperature and CO₂ were maintained at 37° C. and 5%, respectively, with a pressure of 120/80 mmHg. Primary human microvascular endothelial cells (1° ECs) cultured with VascuLife Endothelial Medium served as control and were loaded as per the first day injection for EPC/MesoT cells.

Intravital microscopy was performed to validate recellularized scaffold integrity using a Zeiss inverted microscope after perfusion with carbogen-gassed PBS at 37° C. FITC-dextran retention and vascular integrity was detected using real-time fluorescence as previously described (Kress et al., 2018). Functional endothelium lining the vascular tree was confirmed via acetylated LDL uptake (Invitrogen, L3484); nuclei were counterstained with NucBlue™ Live ReadyProbes™ (Invitrogen, R37605). Metabolically active cells lining the scaffold vasculature were identified by injecting 1 mg/ml of MTT (Serva, 20395) in cell culture media for 90 min followed by aspiration and scaffold washing. Recellularized scaffolds were prepared for immunofluorescence analysis by fixation in 4% paraformaldehyde at RT for 2 hours then embedded in paraffin. Deparaffinized sections (5 uM) were dehydrated and stained with specific antibodies. All primary antibodies were incubated over night at 4° C. followed by conjugated secondary antibodies for 1 hour at RT. Fluorescence was visualized on a Keyence BZ-9000 fluorescence microscope. All antibodies are listed in FIG. S7, Table S1. For light sheet microscopy (LSM) of cellularized scaffolds, tissue was fixed and then stained with primary antibodies for CD31 (Dako, M0823) and NG2 (Abcam, ab83508) at 4° C. overnight. Conjugated secondary antibodies were then incubated at room temperature for 1 hour followed by optical clearing as previously described (Brede et al., 2012).

### Graft Implantation by Anastomosis

Graft implantation into the abdominal region was conducted in female NIH-Foxn1 [symbol] rats (age: 8 weeks, obtained from Charles River). Anesthesia was induced via isoflurane with the intraoperative analgesia Rimadyl® (5 mg/kg subcutaneously, Zoetis, 779-358). After the abdominal cavity was opened by a median laparotomy, the infrarenal aorta abdominalis and the infrahepatic vena cava were isolated from surrounding tissues. Proximal and distal vessels were clamped and side-to-end anastomosis of the scaffold artery to the aorta abdominalis and vein to the vena cava was performed. Following patency examination and perfusion of blood from the host circulatory system, the abdominal cavity was closed occluding the abdominal musculature and skin was sutured. Grafts were harvested after 3 days for further analysis.

### Animal Use and Welfare Compliance

Animal studies and care were conducted in accordance with the Institutional Animal Care and Use Committee (IACUC) at the University of Georgia and FELASA, WHO (WHO-TRS978 Annex3), and FDA (FDA-OCTGT Preclinical Guidance) after approval from the institutional animal protection board (registration reference number #2532-2-12, Ethics Committee of the District of Unterfranken, Würzburg, Germany).

### Statistical Analysis

qRT-PCR samples were done in technical triplicate and error bars calculated in Microsoft Excel. All other assays and statistical measurements were done in GraphPad Prism version 7.0b for Mac (GraphPad Software, La Jolla Calif. USA). SMC contraction assay used one-tailed t-test, Holm-Sidak method to determine statistical significance with alpha=0.05. For carbachol vs. control, the t ratios for 10, 20 and 30 min, respectively, were 5.551, 5.46 and 5.165 with df=38. For KCl vs. control, the t ratios for 10, 20 and 30 min, respectively, were 7.62, 7.318 and 5.581 with df=38. p-values were all <0.0001. All error bars are standard error of the mean (SEM) with n=20 for all time points and groups. TEER significance was determined by comparison against 1° ECs using two-way analysis of variance (ANOVA). Dunnett's test corrected for multiple comparisons with 95% confidence interval (CI), alpha=0.05, F-value=9.376, df=2. Error bars are SEM with n=3 for all measurements. For +VEGF+SB d28 sample vs. 1° ECs, p-value=0.0027. FITC diffusion significance was determined via two-way ANOVA with 1 ECs acting as control for measurements. Control (no cells) were graphed only for visualization. Dunnett's test corrected for multiple comparisons with 95% CI, alpha=0.05, F-value=1.605, df=2. No statistical significance was determined. Errors bars are SEM with n=3 for all measurements. For population doublings, simple linear regression analysis showed a coefficient of determination (R²)=0.9774. Experiment n=3 done in technical triplicate. Error bars are SEM. Cell cycle analysis for p4 (n=4) and p9 (n=3) were all done in technical triplicate; error bars are SEM. Clonal multipotency analysis was determined in technical triplicate by counting positive cells with ImageJ and plotting the average (n=14).

### Results

Results

A hPSC-Derived Cell of the Mesothelium Lineage with Vascular Characteristics

Splanchnic mesoderm (SplM), also referred to herein as Is11+pluripotent cells (IMPs) or alternatively IMPs/SplM) is a transient progenitor population that arises during embryonic heart formation. Developmentally, SplM is specified via Wnt and BMP4 signaling dynamics and is the primitive precursor cell type of epicardium and cardiovascular lineages (Klaus et al., 2012). Equivalent cells can be generated at high efficiency from hPSCs in chemically defined media (CDM) supplemented with Wnt3a and BMP4 (FIG. M1A; FIGS. S1A-S1D). These cells have epithelia characteristics (ZO1⁺, αSMA⁺, VIM⁻)(FIG. M1B), express transcription factors that are characteristic of SplM including ISL1, NKX2.5 and GATA4 (FIGS. M1B and M1C; FIGS. S1A and S1B) and show decreased levels of pluripotency markers NANOG, OCT4 and SOX2 (FIGS. S1C and S1D).

Retinoic acid (RA) signaling and synthesis plays a crucial role in the specification and identification of mesothelium (Iyer et al., 2015; Kikuchi et al., 2011; Perez-Pomares and de la Pompa, 2011; Witty et al., 2014) from hPSC-derived mesoderm as well as during development. While characterizing our hPSC-derived SplM, we found that treatment with all-trans RA promoted a morphological transformation (FIG. M1B). RA treatment downregulated SplM markers (ISL1, NKX2.5) (FIGS. M1B and M1C) and promoted an EMT, as shown by loss of ZO1 and increased vimentin and αSMA expression (FIG. M1B). The RNA-seq signature of RA-treated cells was then compared to that of human and mouse tissues to identify the lineage of these cells (FIG. M1A). Hierarchical clustering analysis of RNA-seq data showed that RA-treated SplM clustered with primary human epicardium and mouse mesothelium isolated from heart, liver, lung and gut (FIG. M1D), suggesting that it belongs to the mesothelium lineage (MesoT). Although MesoTs exhibit characteristics of embryonic mesothelium at the molecular level such as the expression of transcription factors WT1, TBX18 and TCF21 (FIGS. M1B and M1C; FIGS. S1E-S1G) they also have mesenchymal characteristics (αSMA⁺, VIM⁺, ZO1⁻) (FIG. M1B). This contrasts with the typical epithelial characteristics of mesothelium but is reminiscent of mesothelium-derived mesenchymal cells that invade the underlying tissue during organogenesis (Asahina et al., 2009; Que et al., 2008; Smith et al., 2011; Wilm et al., 2005).

To determine whether MesoTs are descendants of visceral mesothelium, we repeated the differentiation of SplM in CDM supplemented with Wnt3a, BMP4 and RA but in the absence of factors known to promote EMT (Activin A and Fgf2) (FIG. S2A). This set of conditions generated epithelial cells that expressed mesothelium markers (FIGS. S2B and S2C) and were designated as “mesothelium-like cells” (MLCs). Once Activin A and Fgf2 signaling was restored, MLCs transitioned through an EMT and towards a phenotype reminiscent of MesoTs at the molecular and cellular level (FIG. S2C). These results are consistent with the development of hPSC-derived SplM along the mesothelium lineage (Nagai et al., 2013; Tian et al., 2015); first through an epithelial state (MLCs) followed by a migratory state (MesoTs).

Since mesothelium-derived cells have been implicated in vascular development during embryogenesis (Rinkevich et al., 2012; Zangi et al., 2013), we sought to obtain corroborative evidence that EPC/MesoT cells have vascular potential by characterizing their epigenetic signature. We identified a MesoT-specific CpG methylation signature that is non-overlapping with corresponding signatures for SplM, hPSC-derived cardiomyocytes (Laflamme et al., 2007) and hPSCs. A cohort of 1846 methylated CpGs were identified that fulfilled this condition (FIG. S3A). This signature was used to screen an expanded panel of DNA methylation datasets including 30 primary human tissues and primary cell samples. This approach showed that primary SMCs, primary ECs and umbilical cord cells have a similar methylation signature to EPC/MesoT cells (FIG. M2A). This indicates that EPC/MesoT cells have epigenetic marks consistent with being part of the vascular lineage.

If MesoTs/EPCs are precursors for either SMCs or ECs it would be anticipated that enhancers required for specification of these vascular lineages would be in a H3K4me1^{high} H3K27ac^{low} ‘primed’ state (Creyghton et al., 2010; Rada-Iglesias et al., 2011) in MesoTs/EPCs (FIG. M2B). To address this, the epigenetic state of active enhancers (H3K27ac^{high}) linked to transcriptionally upregulated gene sets in primary SMCs and ECs were analyzed in MesoTs by ChIP-seq (FIG. M2C; Table S1). These results show that active enhancers linked to vascular transcriptional programs are in a primed state in EPC/MesoT cells, in contrast to the active state in SMCs and ECs (FIG. M2D). A similar analysis was performed where cardiomyocytes (CMs) were compared to EPC/MesoT cells. Here, CM-specific enhancers were not primed in MesoTs/EPCs (FIG. S3B) indicating MesoT cells are not competent for CM differentiation. Gene ontology analysis shows that epigenetically-primed (H3K4me1^{high} H3K27ac^{low}) enhancers in MesoTs are linked to genes that are significantly enriched for functions in vascular development and activity (FIGS. S3C and S3D). Since epigenetic analysis pointed towards MesoTs being part of the vascular lineage, a more focused RNA-seq analysis was also performed. Principal component analysis (PCA) of RNA-seq data shows that MesoT transcriptome is not only similar to MLCs and primary epicardium, but also clusters with vascular cells such as SMCs and endothelial progenitor cells (EPCs) (FIG. M2E). Together, these results are consistent with the hypothesis that MesoTs are of the vascular lineage and are precursors for SMCs and ECs.

### MesoTs/EPCs are Multipotent Vascular Progenitor Cells

With global transcript and epigenetic analysis suggesting EPC/MesoT cells as vascular progenitors of mesothelial origin, we next sought to evaluate the differentiation potential of these cells in vitro. MesoT cells were treated with PDGF-BB or with a combination of VEGF and the TGFβ superfamily type 1 activin receptor-like kinase inhibitor SB431542, conditions known to promote the differentiation of mesoderm to SMC (Mellgren et al., 2008; Rinkevich et al., 2012) and EC (James et al., 2010) identities, respectively. PDGF-BB treatment of MesoTs yielded cultures in which ˜85% cells expressed the SMC markers calponin, alpha smooth muscle actin (αSMA), and myosin heavy chain 11 (MYH11)(FIGS. M3A and M3B). The amplification in cell number as hPSCs transitioned to SMCs through a EPC/MesoT progenitor state is approximately 30-fold (FIG. S4A). Although MesoTs are αSMA⁺ they are distinguished from their downstream derivates, SMCs and Fibroblasts, based on their differential expression of markers such as calponin, MYH11, DDR2, and WT1 (FIG. M3A; FIGS. S4B and S4C). Treatment with the acetylcholine receptor agonist carbachol and the membrane depolarizing agent KCl both triggered cell contraction within 30 minutes (FIGS. M3C and M3D), responses expected of SMCs. In contrast, hESCs showed no contractile activity under these conditions (FIG. S4D). These observations indicate that MesoTs/EPCs are capable of efficiently generating functional SMCs when treated with PDGF-BB.

Treatment of MesoTs/EPCs with VEGF and SB431542 uniformly increased the expression of endothelial markers von Willebrand factor (vWF), CD31, and VE-cadherin (FIGS. M3E and M3F). Endothelial cell markers such as vWF and CD31 are not expressed in MesoTs or in SMCs (FIGS. S4B, S4E, and S4F). Functional characterization of VEGF/SB-treated cells was first evaluated using trans-endothelial electrical resistance (TEER) assays after plating cells on a transwell system coated with a collagen IV-fibronectin matrix. Here, TEER of MesoTs treated with SB/VEGF increased over 28 days and was comparable or greater than that observed for primary human microvascular endothelial cells (FIG. M3G). FITC-dextran diffusion was assessed across cell monolayers treated with VEGF/SB. It was expected that if VEGF/SB treated MesoTs generated selectively permeable endothelial cells they would resist diffusion of FITC-dextran (40 kDa) across the monolayer. As expected, primary endothelial cell monolayers allowed only low levels of label to diffuse through the cellular layer. The extent of FITC-dextran retention was even greater in VEGF alone or VEGF/SB treated EPC/MesoT cells (FIG. M3H), consistent with formation of a tight epithelial barrier. When cultured on a collagen IV-fibronectin-coated transwell insert (FIG. M31), ECs generated from MesoTs/EPCs formed a layer of cells expressing the tight junction protein ZO1 (FIG. M3J and FIG. S4G). When examined by transmission electron microscopy, cells exhibited typical endothelial architecture with tight junctions (FIG. M3K). Together, these data show that functional endothelial cells can be generated from EPC/MesoT progenitors at high efficiency. The overall cell amplification as hPSCs differentiate to ECs through a EPC/MesoT intermediate is ˜50-fold (FIG. S4A). The ˜30-fold amplification to SMCs and 50-fold amplification to ECs indicates that differentiation of MesoT progenitor cells results in significant amplification of vascular lineages.

To obtain evidence that MesoT cells are multipotent vascular progenitors it was necessary to establish culture conditions in which EPC/MesoT cells could be amplified for approximately 15-20 days to allow for clonal amplification. CDM did not support maintenance and proliferation of MesoT cells over this timeframe but supplementation with 10% fetal bovine serum supported maintenance for up to 9 passages. During this period, MesoTs retained their proliferative capacity (FIG. M4A), cell cycle properties (FIGS. M4B-M4D), and global transcriptome profile (FIG. M2E). By interrogating RNA-seq data we identified several cell surface markers expressed on the surface of MesoTs/EPCs that were absent on IMPs/SplM, making these markers potentially useful for cell purification and single cell analysis. To confirm the utility of these markers, which include CD44, CD73, and CD105, triple CD44⁺/CD73⁺/CD105⁺ MesoT cells were isolated, sorted into 96-well dishes and, following amplification, differentiated under smooth muscle (+PDGF-BB) and endothelial cell (+VEGF) conditions (FIGS. M4E-M4F). All 14 amplified clones showed SMC (MYH11⁺) and EC (vWF⁺) differentiation capacity under the respective conditions, although individual clones showed different potencies (FIG. M4G). Thus, evidence supports that MesoTs/EPCs are multipotent vascular progenitor cells, confirming predictions made from the earlier molecular analysis (FIGS. M2 and M3).

It was reasoned that multipotent MesoT cells could give rise to mixed populations of SMCs and ECs. By this rationale, we determined that CDM-Activin A supplemented with VEGF was sufficient to support the differentiation of MesoTs into mixtures of SMCs and ECs (FIG. M4H). Under these conditions, mixtures of SMCs and ECs were generated. When cultured under these conditions for >12 days, mixed populations of SMCs and ECs self-assembled into vessel-like networks (FIG. M4J). The composition and organiation of such ‘vessels’ was evaluated by immunofluorescence (IF) and were shown to be composed of vWF⁺ ECs and αSMA⁺ SMC/pericyte-like cells (FIG. M4J). vWF⁺ cells formed the core of these structures while αSMA⁺ cells were generally located around the periphery. This is an interesting observation because it establishes conditions in which a single progenitor cell (MesoTs/EPCs) can concurrently give rise to SMCs and ECs that unexpectedly self-assemble into a simple vessel structure.

### MesoTs Contribute to Neo-Vascularization During Tissue Repair

To investigate the vascular potential of EPC/MesoT cells in a tissue injury context we used a murine neonatal heart repair model (Porrello et al., 2011). Following resection of the ventricular apex, the neonatal mouse heart undergoes endogenous repair including partial muscle regeneration and neo-vascularization with variable fibrosis (Andersen et al., 2014; Bryant et al., 2015; Porrello et al., 2011). Work in zebrafish suggests that mesothelium-derived cells may be part of such a response by contributing to peri-vascular cells (Kikuchi et al., 2011; Lepilina et al., 2006). To determine whether MesoTs have capacity for vascular repair, they were injected into the pericardial space of P0.5 mouse pups, adjacent to the resected tissue (FIGS. M5A and M5B; FIG. S5A and S5B). 30 days after transplantation mice were sacrificed and cardiac tissue was analyzed by immunohistochemistry on cryosections. Mice receiving saline alone (n=11) or MesoT cells (n=13) revascularized the injured heart. In each heart receiving MesoTs, a major contribution to coronary vessels in repair zones was made by hGolgi⁺ CD31⁺ human cells (FIG. M5C). Hearts that did not receive MesoT cells repaired as expected but were negative for hGolgi antigen (FIG. S5C). These vessels were connected to the host vasculature as indicated by the presence of erythrocytes (FIG. S5D). Further analysis showed that hGolgi⁺ CD31⁺ endothelial cells and hGolgi αSMA⁺ SMC/pericyte-like cells made significant contributions to the neo-vasculature in repair zones (FIG. MSD). These results show that MesoT progenitor cells can differentiate and assemble into functional vessels in this tissue injury model and suggests that they may be useful in a broader range of regenerative approaches.

### Vascularization of Biological Scaffolds by MesoTs: In Vitro and In Vivo Function

The cell culture and in viv experiments presented suggest that EPC/MesoT cells may be a viable alternative to existing approaches for the recellularization of vascular scaffolds used for tissue engineering. To investigate this possibility we used explanted, decellularized rat jejunal segments (FIG. M6A) as a biological scaffold on which to seed MesoT cells in a bioreactor (FIG. S6A). In this model, the rat vasculature remains intact and can be perfused using the afferent artery and efferent vein (FIGS. M6B and M6C). Following the introduction of EPC/MesoT cells through the lumen of the vascular bed, constructs were perfused with medium supporting differentiation to SMCs and ECs (FIGS. M4H-M4J). Perfusion was pulsatile and increased gradually to a physiological pressure of 80-120 mmHg to promote vessel maturation. Constructs were then perfused with 3-(4,5-dimethylthiazol-2-yl)-2,5-diphenyltetrazolium bromide (MTT), a substrate used to evaluate the assembly of metabolically active cells in the vascular network (FIG. M6D). This shows that viable cells broadly lined the jejunal scaffold and that these cells were of human origin (FIG. M6E). No MTT staining was observed in constructs that were not recellularized (FIG. S6B). Analysis of vessels by IF on cryo-sections showed that capillaries were lined with human CD31⁺ cells and that medium (˜100-150 μm) and larger (>150 μm) vessels were lined with CD31⁺ cells and αSMA⁺ cells. The organization of these cells was as expected, with CD31+endothelial cells forming a contiguous layer on the lumenal surface and αSMA⁺ SMCs forming an adjacent layer on the ablumenal side (FIGS. M6F-M6H). The perfusion of decellularized vascular beds with MesoT cells supports their differentiation and self-assembly into vascular structures; this is reminiscent of their behavior in two-dimensional culture (FIGS. M4H-M4J).

To evaluate the barrier function of EPC/MesoT-derived cells in the vascular structures, cellularized constructs were perfused with FITC-dextran and monitored by time lapse imaging. We observed that MesoT-derived vessels retained FITC-dextran (Figure M6I) in contrast to constructs that were sparsely-seeded where rapid diffusion and leakage was observed (FIG. S6C). Scaffolds without cells are not shown because label leaks immediately at the site of infusion and can't be visualized. Endothelial cell function was verified by demonstrating the uptake of acetylated low-density lipoprotein (LDL)(Figure M6I). To determine the extent to which MesoT-derived endothelium lined the vascular tree, we stained perfused constructs with human-specific anti-CD31 antibody. Light sheet microscopy (LSM) showed widespread incorporation of these cells into the vasculature (FIG. M6J; FIGS. S6D and S6E), as expected from our analysis of the frozen sections (FIGS. M6F-M6H). LSM also showed incorporation of NG2⁺ pericyte-like cells into vascular networks (FIG. S6F). This indicates that EPC/MesoT cells can contribute to three vascular lineages and that these three vascular lineages self-assemble into vascular tissue.

To investigate the functionality of EPC/MesoT-populated vascular constructs under physiological conditions, recellularized jejunal scaffolds were connected to the rat circulation by anastomosis (FIG. M6K) for 3 days. Analysis of anastomosed constructs showed that vessels retained gross morphologic integrity with no indication of blood leakage or occlusion (FIG. M6L; FIG. S6G). Further analysis of these vessels by IF following cryo-sectioning showed maintenance of capillaries and larger vessels, in composition and architecture, after anastomosis (FIGS. 6M-6O). These observations show that MesoTs can populate biological scaffolds with cells that assemble into vessels which withstand physiological pressures and conditions, at least over short periods of time.

### DISCUSSION

Several reports have described mesenchymal progenitor descendants of the epicardial lineage (Chong et al., 2011; Gittenberger-de Groot et al., 2010; Rinkevich et al., 2012; Singh and Epstein, 2012), however, embryonic mesothelial layers of different coelomic organs have similar molecular and cellular characteristics (Rinkevich et al., 2012; Winters et al., 2014) (FIG. M1D). Lineage-marked descendants of mesothelium from multiple organs have been isolated and shown to have a similar potency to epicardium-derived cells (EPDCs) (Rinkevich et al., 2012). While there is some debate about the degree to which EPDCs contribute to the endothelium of the coronary vasculature (Pennisi, 2016), several studies indicate some contribution of the epicardium lineage to arterial vessels with the remainder coming from other sources (Cano et al., 2016; Chen et al., 2014a; Katz et al., 2012; Palmquist-Gomes et al., 2018; Perez-Pomares and de la Pompa, 2011). Whether this endothelial contribution comes from EPDCs or another epicardial subtype is currently not known.

While ‘epicardium’ from hPSCs (Iyer et al., 2015; Witty et al., 2014) are potentially generated along similar developmental pathways as MesoT cells, they differ in key respects. First, ‘epicardium’ cells are epithelial and develop through a ‘pro-epicardium’ stage (Witty et al., 2014). Second, hPSC-derived ‘epicardium’ has a propensity for SMC and fibroblast differentiation, with no multipotency or endothelial potential reported (Gittenberger-de Groot et al., 2010). It has been proposed that the pro-epicardium and epicardium are heterogeneous in cell composition (Cao et al., 2016; Katz et al., 2012), making different contributions to the coronary vasculature. It is possible that hPSC-derived ‘epicardium’ and the mesothelium described here, are representative of different mesothelial subtypes but because the developmental pathway for each has not been clearly defined, it is not possible to unequivocally assign MLCs and MesoTs to mesothelium from a specific anatomical location (e.g. the heart). In the absence of specific information to address this issue, we have designated MLCs and MesoT cells as being of ‘mesothelial origin’. We hypothesize from a developmental standpoint, that MLCs represent mesothelium and that MesoTs are their vasculogenic mesenchymal descendants (Chong et al., 2011; Rinkevich et al., 2012; Singh and Epstein, 2012).

The differentiation of hPSCs along the mesothelium lineage described here is consistent with the generation of this cell type during embryonic development. The approach described in this report shows that IMP/SplM progenitors are efficiently specified along the mesothelium lineage by a combination of BMP, Wnt and RA signaling. The mechanism of lineage specification by these signaling pathways is not understood and will be a subject of future investigation. In the future, it will be necessary to evaluate the relationship between MLCs and MesoT cells further. For example; is the developmental potential of MesoTs/EPCs also exhibited by MLCs. The incorporation of MesoT-derived pericytes into vascular constructs suggests that they can be directly generated from MesoT cells in culture. Further work will be required to establish if MesoT cells can generate pericytes at high-efficiency by directed differentiation.

Following the plating of EPC/MesoT cells under specific signaling conditions, a mixture of SMCs and ECs are generated which self-assemble into tube like networks. This assay is reminiscent of tube forming assays used to characterize EC progenitors (Alphonse et al., 2015; Prasain et al., 2014). Here, ECs migrate over a two-dimensional matrix and assemble into tube-like structures. In contrast to EC progenitors, that give rise to tubes comprised solely of endothelial cells, EPC/MesoT cells generate invested structures composed of SMCs and ECs. It is assumed that part of the tube assembly mechanism described in this report is similar to that described for EC progenitors with the additional complexity of SMCs superimposed into the system. This general process has been described previously where mixed populations of SMCs and ECs are plated simultaneously and give rise to invested tube-like structures (Marchand et al., 2014). Understanding the assembly of MesoT-derived SMCs and ECs into tube structures will be important to harness the full utility of MesoT cells for translational purposes. The self-assembly of MesoT-derived cells into tube structures in a two-dimensional culture system suggested that this may also occur in a three-dimensional system, supported by shear stress from perfusion. This was confirmed using decellularized vascular scaffolds that were shown to be stable and functional for over 28 days. In the future, it will be necessary to optimize numerous variables in vessel construction using MesoT cells including perfusion rates and pressures, seeding density, media composition and scaffold composition.

In this series of experiments, a mesenchymal cell of the mesothelium lineage that has multipotent vascular potential has been characterized. Even though extensive efforts were made, cardiomyocytes could not be generated from MesoT cells, indicating that this progenitor does not have broad spectrum cardiovascular lineage potential. This is supported by epigenetic analysis in this study. Epigenetic characterization supports our conclusions that MesoT cells are a multipotent vascular progenitor based on the ‘priming’ signature observed at enhancers used by vascular lineages and by the DNA methylation profile linking them to vascular cell types. We are not aware of any other reported cell type from the mesothelium lineage that has the multipotent, vascular progenitor characteristics of that described here for EPC/MesoT cells. hPSC- and tissue-derived vascular cell types, including SMCs and ECs, have been used for the development of cell therapeutics and tissue engineering (Doi et al., 2017; Ren et al., 2015), providing proof of concept for this strategy. However, barriers to using these cell types in vascular repair scenarios exist and need to be considered if PSC-derived vascular cells are to become clinically viable. For example, fully differentiated SMCs and ECs have not yet been shown to efficiently incorporate into new vasculature in a tissue repair model following transplantation. This could be related to limitations of mature cells to integrate into remodeling tissue or an inability to assemble into neo-vessels in a repair environment. In tissue engineering, combinations of SMCs, ECs and mesenchymal stem cells (MSCs) are frequently used to seed vascular scaffolds, but this process is complicated, and the long-term efficacy of such constructs are unclear (Drews et al., 2017; Lee et al., 2016; Ren et al., 2015; Villalona et al., 2010). The results here suggest that vascular progenitor cells may be a solution to the problems associated with using mature vascular cell types. In support of this idea, circulating EPCs first identified In vivo (Alphonse et al., 2015; Asahara et al., 1997), but also recently derived from hPSCs, show functional efficacy by contributing to new vasculature in a diabetic retinopathy model (Prasain et al., 2014). Although EPCs have promising clinical utility, their potency is restricted to the endothelial lineage. These limitations do not apply to MesoT cells because of their vasculogenic, multipotent properties.

To develop strategies for use of MesoT cells in vascular engineering it will be necessary to apply them to vessels that have utility in a transplantation context. Instead of using a vascular tree, decellularized vessels or fabricated scaffolds will be utilized on which to seed MesoT cells. Fine-tuning of seeding and perfusion conditions will then be required to obtain vessels with suitable characteristics for transplantation purposes. For example, the generation of venous and arterial vessels. The properties of MesoT cells described in this report indicate that they offer an important option for vessel engineering in a wide-range of contexts.

### REFERENCES

Alphonse, R. S., Vadivel, A., Zong, S., McConaghy, S., Ohis, R., Yoder, M. C., and Thebaud, B. (2015). The isolation and culture of endothelial colony-forming cells from human and rat lungs. Nat Protoc 10, 1697-1708.
Andersen, D. C., Ganesalingam, S., Jensen, C. H., and Sheikh, S. P. (2014). Do neonatal mouse hearts regenerate following heart apex resection? Stem Cell Reports 2, 406-413.
Asahara, T., Murohara, T., Sullivan, A., Silver, M., van der Zee, R., Li, T., Witzenbichler, B., Schatteman, G., and Isner, J. M. (1997). Isolation of Putative Progenitor Endothelial Cells for Angiogenesis. Science 275, 964-966.
Asahina, K., Tsai, S. Y., Li, P., Ishii, M., Maxson, R. E., Jr., Sucov, H. M., and Tsukamoto, H. (2009). Mesenchymal origin of hepatic stellate cells, submesothelial cells, and perivascular mesenchymal cells during mouse liver development. Hepatology 49, 998-1011.
Berger, R. P., Sun, Y. H., Kulik, M., Lee, J. K., Nairn, A. V., Moremen, K. W., Pierce, M., and Dalton, S. (2016). ST8SIA4-Dependent Polysialylation is Part of a Developmental Program Required for Germ Layer Formation from Human Pluripotent Stem Cells. Stem Cells 34, 1742-1752.
Brede. C., Friedrich, M., Jordin-Garrote, A.-L., Riedel. S. S., Bauerlein, C. A., Heinze, K. G., Bopp, T., Schulz, S., Mottok, A., Kiesel, C., Mattenheimer, K., Ritz, M., Krosigk, von, V., Rosenwald, A., Einsele, H., Negrin, R. S., Harms, G. S., Beilhack, A., 2012. Mapping immune processes in intact tissues at cellular resolution. J. Clin. Invest. 122, 4439-4446. doi:10.1172/JC165100
Bryant, D. M., O'Meara, C. C., Ho, N. N., Gannon, J., Cai, L., and Lee, R. T. (2015). A systematic analysis of neonatal mouse heart regeneration after apical resection. J Mol Cell Cardiol 79, 315-318.
Cano, E., Carmona, R., and Munoz-Chapuli, R. (2013). Wt1-expressing progenitors contribute to multiple tissues in the developing lung. Am J Physiol Lung Cell Mol Physiol 305, L322-L332.
Cano, E., Cannona, R., Ruiz-Villalba, A., Rojas, A., Chan, Y. Y., Wagner, K. D., Wagner, N., Hastie, N. D., Munoz-Chapuli, R., and Perez-Pomares, J. M. (2016). Extracardiac septum transversum-/proepicardial endothelial cells pattern embryonic coronary arterio-venous connections. Proc Nal Acad Sci USA 113, 656-661.
Cao, J., Navis, A., Cox, B. D., Dickson, A. L., Gemberling, M., Karra, R., Bagnat, M., and Poss, K. D. (2016). Single epicardial cell transcriptome sequencing identifies Caveolin 1 as an essential factor in zebrafish heart regeneration, Development 143, 232-243.
Chen, H. I., Sharma, B., Akerberg, B. N., Numi, H. J., Kivela, R., Saharinen, P., Aghajanian, H., McKay, A. S., Bogard, P. E., Chang, A. H., et al. (2014a). The sinus venosus contributes to coronary vasculature through VEGFC-stimulated angiogenesis. Development 141, 4500-4512.
Chen, T.-W., Li, H.-P., Lee, C.-C., Gan, R.-C., Huang, P.-J., Wu, T. H., Lee, C.-Y., Chang, Y.-F., and Tang, P. (2014b). ChIPseek, a web-based analysis tool for ChIP data. BMC Genomics 15, 539.
Cheung, C., Bernardo, A. S., Trotter, M. W., Pedersen, R. A., and Sinha, S. (2012). Generation of human vascular smooth muscle subtypes provides insight into embryological origin-dependent disease susceptibility. Nat Biotechnol 30, 165-173.
Chong, J. J., Chandrakanthan, V., Xaymardan, M., Asli, N. S., Li. J., Ahmed. I., Heffernan. C., Menon, M. K., Scarlett, C. J., Rashidianfar, A., et al. (2011). Adult cardiac-resident MSC-like stem cells with a proepicardial origin. Cell Stem Cell 9, 527-540.
Cliff, T. S., Wu, T., Boward, B. R., Yin, A., Yin, H., Glushka, J. N., Prestegaard, J. H., and Dalton, S. (2017). MYC Controls Human Pluripotent Stem Cell Fate Decisions through Regulation of Metabolic Flux. Cell Stem Cell 21, 502-516 e509.
Colunga, T., and Dalton, S. (2018). Building Blood Vessels with Vascular Progenitor Cells. Trends Mol Med 24, 630-641.

Creyghton, M. P., Cheng, A. W., Welstead, G. G., Kooistra, T., Carey, B. W., Steine, E. J., Hanna, J., Lodato, M. A., Frampton, G. M., Sharp, P. A., et al. (2010). Histone H3K27ac separates active from poised enhancers and predicts developmental state. Proc Natl Acad Sci USA 107, 21931-21936.
Dixit, R., Ai, X., and Fine, A. (2013). Derivation of lung mesenchymal lineages from the fetal mesothelium requires hedgehog signaling for mesothelial cell entry. Development 140, 4398-4406.
Doi, R., Tsuchiya, T., Mitsutake, N., Nishimura, S., Matsuu-Matsuyama, M., Nakazawa, Y., Ogi, T., Akita, S., Yukawa, H., Baba, Y., et al. (2017). Transplantation of bioengineered rat lungs recellularized with endothelial and adipose-derived stromal cells. Sci Rep 7, 8447.
Drews, J. D., Miyachi, H., and Shinoka, T. (2017). Tissue-engineered vascular grafts for congenital cardiac disease: Clinical experience and current status. Trends Cardiovasc Med 27, 521-531.
Elmadbouh, L, Chen, Y., Louedec, L., Silberman, S., Pouzet, B., Meilhac, O., and Michel, J. B. (2005). Mesothelial cell transplantation in the infarct scar induces neovascularization and improves heart function. Cardiovasc Res 68, 307-317.
Gittenberger-de Groot, A. C., Winter, E. M., and Poelmann, R. E. (2010). Epicardium-derived cells (EPDCs) in development, cardiac disease and repair of ischemia. J Cell Mol Med 14, 1056-1060.
Iyer, D., Gambardella, L., Bernard, W. G., Serrano, F., Mascetti, V. L., Pedersen, R. A., Talasila, A., and Sinha, S. (2015). Robust derivation of epicardium and its differentiated smooth muscle cell progeny from human pluripotent stem cells. Development 142, 1528-1541.
James, D., Hyung-song, N., Seandel, M., Nolan, D., Janovitz, T., Tomishima, M., Studer, L., Lee, G., Lyden, D., Benezra, R., et al. (2010). Expansion and maintenance of human embryonic stem cell-derived endothelial cells by TGFβ inhibition is Id1 dependent. Nat Biotechnol 28, 161-166.
Katz, T. C., Singh, M. K., Degenhardt, K., Rivera-Feliciano, J., Johnson, R. L., Epstein, J. A., and Tabin, C. J. (2012). Distinct compartments of the proepicardial organ give rise to coronary vascular endothelial cells. Dev Cell 22, 639-650.
Kikuchi, K., Holdway, I. E., Major, R. J., Blum, N., Dahn, R. D., Begemann, G., and Poss, K. D. (2011). Retinoic acid production by endocardium and epicardium is an injury response essential for zebrafish heart regeneration. Dev Cell 20, 397-404.
Kim, D., Pertea, G., Trapnell, C., Pimentel, H., Kelley, R., and Salzberg, S. L. (2013). TopHat2: accurate alignment of transcriptomes in the presence of insertions, deletions and gene fusions. Genome Biol 14, R36.
Klaus, A., Muller, M., Schulz, H., Saga, Y., Martin, J. F., and Birchmeier, W. (2012). Wnt/beta-catenin and Bmp signals control distinct sets of transcription factors in cardiac progenitor cells. Proc Nat Acad Sci USA 109, 10921-10926.
Kress, S., Baur, J., Otto, C., Burkard, N., Braspenning, J., Walles. H., Nickel, J., and Metzger, M. (2018). Evaluation of a miniaturized biologically vascularized scaffold in vitro and in vivo, Sci Rep 8, 4719.
Laflamme, M. A., Chen, K. Y., Naumova, A. V., Muskheli, V., Fugate, J. A., Dupras, S. K., Reinecke, H., Xu, C., Hassanipour, M., Police, S., et al. (2007). Cardiomyocytes derived from human embryonic stem cells in pro-survival factors enhance function of infarcted rat hearts. Nat Biotechnol 25, 1015-1024.
Langfelder, P., and Horvath, S. (2008). WGCNA: an R package for weighted correlation network analysis. BMC Bioinformatics 9, 559.
Langmead, B., Trapnell, C., Pop, M., and Salzberg, S. L. (2009). Ultrafast and memory-efficient alignment of short DNA sequences to the human genome. Genome Biol 10, R25.
Law, C. W., Chen, Y., Shi, W., and Smyth, G. K. (2014). voom: precision weights unlock linear model analysis tools for RNA-seq read counts. Genome Biol 15, R29.
Lee, Y.-U., Mahler, N., Best, C. A., Tara, S., Sugiura, T., Lee, A. Y., Yi, T., Hibino, N., Shinoka, T., and Breuer, C. (2016). Rational design of an improved tissue-engineered vascular graft: determining the optimal cell dose and incubation time. Regen Med 11, 159-167.
Lepilina, A., Coon, A. N., Kikuchi, K., Holdway, J. E., Roberts, R. W., Burns, C. G., and Poss, K. D. (2006). A dynamic epicardial injury response supports progenitor cell activity during zebrafish heart regeneration. Cell 127, 607-619.
Li, H., Handsaker, B., Wysoker, A., Fennell, T, Ruan, J., Homer, N, Marth, G., Abecasis, G., Durbin, R., and Genome Project Data Processing. S. (2009). The sequence alignment/map format and SAMtools. Bioinformatics 25, 2078-2079.
Liao, Y., Smyth, G. K., and Shi, W. (2013). The Subread aligner: fast, accurate and scalable read mapping by seed-and-vote. Nucleic Acids Res 41, e108.
Liu, Q., Huang, X., Oh, J. H., Lin, R. Z., Duan, S., Yu, Y., Yang, R., Qiu, J., Melero-Martin, J. M., Pu, W. T., et al. (2014). Epicardium-to-fat transition in injured heart. Cell Res 24, 1367-1369.
Lucas, P. A. (2007). Stem Cells for Mesothelial Repair: An Understudied Modality. Int J Artif Organs 30, 550-556.
Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140.
Maksimovic, J., Gordon, L., and Oshlack, A. (2012). SWAN: Subset-quantile within array normalization for illumina infinium HumanMethylation450 BeadChips. Genome Biol 13, R44.
Marchand, M., Anderson, E. K., Phadnis, S. M., Longaker, M. T., Cooke, J. P., Chen, B., Reijo Pera R. A., 2014. Concurrent generation of functional smooth muscle and endothelial cells via a vascular progenitor. Stem Cells Transl Med 3, 91-97. doi:10.5966/sctm.2013-0124.
McCarthy, D. J., Chen, Y., and Smyth, G. K. (2012). Differential expression analysis of multifactor RNA-Seq experiments with respect to biological variation. Nucleic Acids Res 40, 4288-4297.
Mellgren, A. M., Smith, C. L., Olsen, G. S., Eskiocak, B., Zhou. B., Kazi, M. N., Ruiz, F. R., Pu, W. T., and Tallquist, M. D. (2008). Platelet-derived growth factor receptor beta signaling is required for efficient epicardial cell migration and development of two distinct coronary vascular smooth muscle cell populations. Circ Res 103, 1393-1401.
Mi, H., Huang, X. Muruganujan, A., Tang, H., Mills, C., Kang, D., and Thomas, P. D. (2017). PANTHER version 11: expanded annotation data from Gene Ontology and Reactome pathways, and data analysis tool enhancements. Nucleic Acids Res 45, D183-D189.
Motherwell, J. M., Azimi, M. S., Spicer, K., Alves, N. G., Hodges, N. A., Breslin, J. W., Katakam, P. V. G., and Murfee. W. L. (2017). Evaluation of Arteriolar Smooth Muscle Cell Function in an Ex Vivo Microvascular Network Model. Sci Rep 7, 2195.
Mutsaers, S. E., and Wilkosz, S. (2007). Structure and Function of Mesothelial Cells. In Peritoneal Carcinomatosis: A Multidisciplinary Approach, W. P. Ceelen, ed. (Boston, Mass.: Springer US), pp. 1-19.
Nagai, H., Chew, S. H., Okazaki, Y., Funahashi, S., Namba, T., Kato, T., Enomoto, A., Jiang, L., Akatsuka, S., and Toyokuni, S. (2013). Metamorphosis of mesothelial cells with active horizontal motility in tissue culture. Sci Rep 3, 1144.
Palmquist-Gomes, P., Antonio Guadix, J., and Perez-Pomares, J. M. (2018). Avian embryonic coronary arterio-venous patterning involves the contribution of different endothelial and endocardial cell populations. Dev Dyn 247, 686-698.
Patsch, C., Challet-Meylan, L, Thoma, E. C., Urich, E., Heckel, T., O'Sullivan, J. F., Grainger, S. J., Kapp, F. G., Sun, L., Christensen, K., et al. (2015). Generation of vascular endothelial and smooth muscle cells from human pluripotent stem cells. Nat Cell Biol 17, 994-1003.
Pennisi, D. J. (2016). Towards consensus on coronary vessel development: Coronary arterial endothelial cells derive primarily from the sinus venosus during embryogenesis. Circ Res 118, 221-232.
Perez-Pomares, J. M., and de la Pompa, J. L. (2011). Signaling during epicardium and coronary vessel development. Circ Res 109, 1429-1442.
Porrello, E. R., Mahmoud, A. I., Simpson, E., Hill, J. A., Richardson, J. A., Olson, E. N., and Sadek, H. A. (2011). Transient regenerative potential of the neonatal mouse heart. Science 331, 1078-1080.
Prasain, N., Lee, M. R., Vemula, S., Meador, J. L., Yoshimoto. M., Ferkowicz, M. J., Fett. A., Gupta, M., Rapp, B. M., Saadatzadeh, M. R., et al. (2014). Differentiation of human pluripotent stem cells to cells similar to cord-blood endothelial colony-forming cells. Nat Biotechnol 32, 1151-1157.
Que, J., Wilm, B., Hasegawa, H., Wang, F., Bader, D., and Hogan, B. L. M. (2008). Mesothelium Contributes to Vascular Smooth Muscle and Mesenchyme during Lung Development. Proc Natl Acad Sci USA 105, 16626-16630.
R Core Team. (2013). R: A language and environment for statistical computing. R Foundation for Statistical Computing, Vienna, Austria. URL http://www.R-project.org/.
Rada-iglesias, A., Bajpai, R., Swigut, T., Brugmann, S. A., Flynn, R. A., and Wysocka, J. (2011). A unique chromatin signature uncovers early developmental enhancers in humans. Nature 470, 279-283.
Ramirez, F., Ryan, D. P., Gruning, B., Bhardwaj, V., Kilpert, F., Richter, A. S., Heyne, S., Dundar, F., and Manke, T. (2016). deepTools2: a next generation web server for deep-sequencing data analysis. Nucleic Acids Res 44, W160-W165.
Ren, X., Moser, P. T., Gilpin, S. E., Okamoto, T., Wu, T., Tapias, L. F., Mercier, F. E., Xiong, L., Ghawi, R., Scadden, D. T., et al. (2015). Engineering pulmonary vasculature in decellularized rat and human lungs. Nat Biotechnol 33, 1097-1102.
Rinkevich, Y., Mori, T., Sahoo, D., Xu, P. X, Bermingham, J. R., Jr., and Weissman. I. L. (2012). Identification and prospective isolation of a mesothelial precursor lineage giving rise to smooth muscle cells and fibroblasts for mammalian internal organs, and their vasculature. Nat Cell Biol 14, 1251-1260.
Ritchie, M. E., Phipson, B., Wu, D., Hu, Y., Law, C. W., Shi, W., and Smyth, G. K. (2015), limma powers differential expression analyses for RNA-sequencing and microarray studies. Nucleic Acids Res 43, e47.
Robinson, M. D., McCarthy, D. J., and Smyth, G. K. (2010). edgeR: a Bioconductor package for differential expression analysis of digital gene expression data. Bioinformatics 26, 139-140.
Robinson, M. D., and Oshlack, A. (2010). A scaling normalization method for differential expression analysis of RNA-seq data. Genome Biol 11, R25.
RStudio Team. (2015). RStudio: Integrated Development for R. RStudio, Inc. Boston, Mass.
Shelton. E. L., and Bader, D. M. (2012). Thymosin beta4 mobilizes mesothelial cells for blood vessel repair. Ann N Y Acad Sci 1269, 125-130.
Si-Tayeb, K., Noto, F. K., Sepac, A., Sedlic, F., Bosnjak, Z. J., Lough, J. W., and Duncan, S. A. (2010). Generation of human induced pluripotent stem cells by simple transient transfection of plasmid DNA encoding reprogramming factors. BMC Dev Biol 10, 81.
Singh, A. M., Sun, Y., Li, L., Zhang, W., Wu, T., Zhao, S., Qin, Z, and Dalton, S. (2015). Cell-Cycle Control of Bivalent Epigenetic Domains Regulates the Exit from Pluripotency. Stem Cell Reports S, 323-336.
Singh, M. K., and Epstein, J. A. (2012). Epicardium-derived cardiac mesenchymal stem cells: expanding the outer limit of heart repair. Circ Res 110, 904-906.
Smart, N., Bollini, S., Dube, K. N., Vieira, J. M., Zhou, B., Davidson, S., Yellon, D., Riegler, J., Price, A. N., Lythgoe, M. F., et al. (2011). De novo cardiomyocytes from within the activated adult heart after injury. Nature 474, 640-644.
Smith, C. L., Baek, S. T., Sung, C. Y., and Tallquist, M. D. (2011). Epicardial-derived cell epithelial-to-mesenchymal transition and fate specification require PDGF receptor signaling. Circ Res 108, e15-26.
Smyth, G. K. (2004). Linear models and empirical bayes methods for assessing differential expression in microarray experiments. Stat Appi Genet Mol Biol 3, Article3.
Su, S., Law, C. W., Ah-Cann, C., Asselin-Labat, M. L., Blewitt, M. E., and Ritchie, M. E. (2017). Glimma: interactive graphics for gene expression analysis. Bioinformatics 33, 2050-2052.
Takeuchi, M., Takeuchi, K., Ozawa, Y., Kohara, A., and Mizusawa, H. (2009). Aneuploidy in Immortalized Human Mesenchymal Stem Cells with Non-Random Loss of Chromosome 13 in Culture. In Vitro Cell Dev-An 45, 290-299.
Tian, X., Pu, W. T., and Zhou, B. (2015). Cellular origin and developmental program of coronary angiogenesis. Circ Res 116, 515-530.
van Wijk, B., Gunst, Q. D., Moorman, A. F. M., and Van den Hoff, M. J. B. (2012). Cardiac Regeneration from Activated Epicardium. PLoS ONE 7, 1-14.
Villalona, G. A., Udelsman, B., Duncan, D. R., McGillicuddy, E., Sawh-Martinez, R., Hibino, N., Paínter, C., Mirensky, T., Erickson, B., Shinoka, T., et al. (2010). Cell-Seeding Techniques in Vascular Tissue Engineering. Tissue Eng Part B Rev 16, 341-350.
Wang, L., Schulz, T. C., Sherrer, E. S., Dauphin, D. S., Shin, S., Nelson, A. M., Ware, C. B., Zhan, M., Song, C.-Z., Chen, X., et al. (2007). Self-renewal of human embryonic stem cells requires insulin-like growth factor-1 receptor and ERBB2 receptor signaling. Blood 110, 4111-4119.
Wilm, B., Ipenberg, A., Hastie, N. D., Burch, J. B., and Bader, D. M. (2005). The serosal mesothelium is a major source of smooth muscle cells of the gut vasculature. Development 132, 5317-5328.
Winters, N. I., Williams. A. M., and Bader, D. M. (2014). Resident progenitors, not exogenous migratory cells, generate the majority of visceral mesothelium in organogenesis. Dev Biol 391, 125-132.
Witty, A. D., Mihic, A., Tam, R. Y., Fisher, S. A., Mikryukov, A., Shoichet, M. S., Li, R. K., Kaltman, S. J., and Keller, G. (2014). Generation of the epicardial lineage from human pluripotent stem cells. Nat Biotechnol 32, 1026-1035.
Wixson, S. K., and Smiler, K. L (1997). Anesthesia and Analgesia in Rodents. In Anesthesia and Analgesia in Laboratory Animals, D. F. Kohn, ed. (San Diego: Academic Press), pp. 165-203.
Zangi, L., Lui, K. O., von Gise, A., Ma, Q., Ebina, W., Ptaszek, L. M., Spater. D., Xu, H., Tabebordbar, M., Gorbatov, R., et al. (2013). Modified mRNA directs the fate of heart progenitor cells and induces vascular regeneration after myocardial infarction. Nat Biotechnol 31, 898-907.
Zhou, B., Ma, Q., Rajagopal, S., Wu, S. M., Domian, I., Rivera-Feliciano, J., Jiang, D., von Gise, A., Ikeda, S., Chien, K. R., et al. (2008). Epicardial progenitors contribute to the cardiomyocyte lineage in the developing heart. Nature 454, 109-113.
Zhou, B., and Pu, W. T. (2012). Isolation and characterization of embryonic and adult epicardium and epicardium-derived cells. In Cardiovascular Development Methods in Molecular Biology (Methods and Protocols) (Totowa: Humana Press). pp. 155-168.

1. A method of producing a vascular tissue containing vessel comprising seeding a tubular biomaterial scaffold with an effective number of migratory mesoderm cells (MesoTs) in association with said scaffold and exposing said MesoTs to a differentiation medium which produces endothelial cells, smooth muscle cells and pericytes from said MesoTs, wherein said endothelial cells, smooth muscle cells and pericytes self-assemble in association with said scaffold to produce said vascular vessel.

2. The method according to claim 1 wherein said tubular biomaterial scaffold is a decellularized tubular material obtained by decellularizing tubular tissue obtained from an animal.

3. The method according to claim 2 wherein said animal is a human, non-human primate, pig or cow.

4. The method according to claim 2 or 3 wherein said decellularized tubular material is obtained by exposing tubular tissue obtained from an animal to physical agitation, chemical surfactant removal and enzymatic digestion to disrupt cells and remove proteins, lipids and nucleic acids from the tubular tissue to provide a decellularized vascular scaffold.

5. The method according to claim 1 wherein said tubular biomaterial scaffold comprises a polymer which has been electrospun or 3D printed into a tubular scaffold.

6. The method according to claim 1 wherein said tubular biomaterial scaffold comprises collagen.

7. The method according to claim 6 wherein said tubular material scaffold comprises a collagen and polymer blend.

8. The method according to any of claims 1-7 wherein said differentiation medium comprises at least a minimum essential medium, in combination with an effective amount of a VEGF signaling pathway agonist or VEGF receptor agonist, insulin-like growth factor IGF), fibroblast growth factor 2 (FGF2) and heregulin β.

9. The method according to any of claims 1-8 wherein said differentiation medium comprises DMEM/F12 media supplemented with heregulin β, IGF, FGF2 and VEGF.

10. The method according to any of claims 1-8 wherein said differentiation medium is chemically defined media (CDM) which excludes Activin A and includes an effective amount of a VEGF signaling pathway agonist or VEGF receptor agonist.

11. The method according to any one of claims 1-10 wherein said MesoTs are differentiated in association with said scaffold for a period ranging from 1 week to 6 weeks.

12. The method according to any of claims 1-10 wherein said MesoTs are differentiated in association with said scaffold for a period of 12-14 days to 30 days.

13. The method according to any one of claims 1-10 wherein said MesoTs are seeded onto said scaffold for a period of several hours to 5 days.

14. A cellularized vascular vessel comprising a tubular biomaterial in association with a population of self-assembled endothelial cells, smooth muscle cells and pericytes which have been differentiated from migratory mesoderm cells (MesoTs).

15. The vessel according to claim 14 wherein said tubular biomaterial is obtained by decellularizing tubular tissue from an animal.

16. The vessel according to claim 15 wherein said animal is a human, non-human primate, pig or cow.

17. A kit comprising tubular biomaterial having a diameter of less than 1 mm to 25 cm, a population of MesoTs effective to seed said tubular biomaterial and an effective amount of differentiation medium with instructions for seeding the tubular biomaterial with the MesoTs and differentiating the MesoTs with the differentiation medium into endothelial cells, smooth muscle cells and pericytes which self-assemble into vascular tissue in association with the tubular biomaterial, thus forming a vascular vessel.

18. The kit according to claim 17 wherein said tubular biomaterial is tubular decellularized animal vascular tissue.

19. The kid according to claim 17 wherein said tubular biomaterial is a tube formed from collagen or a mixture of collagen and a polymeric material.

20. A composition comprising between 5×10⁴ and 5×10⁹ MesoT cells in combination with a differentiation medium which excludes Activin A and includes an effective amount of heregulin, IGF, FGF-2 and a VEGF signaling pathway agonist or VEGF receptor agonist.

21. The composition according to claim 20 wherein said differentiation medium is chemically defined media (CDM) excluding Activin A and including an effective amount of a VEGF signaling pathway agonist or VEGF receptor agonist.

22. The composition according to claim 20 or 21 which comprises between 10⁵ and 10⁸ Meso T cells.

23. The composition according to any of claims 20-22 which comprises between 10⁵ and 10⁶ Meso T cells.

24. The composition according to any of claims 20-23 wherein said VEGF signaling pathway agonist or VEGF receptor agonist is rhVEGF-A₁₆₅.

25. The composition according to any of claims 20-24 wherein said composition has been seeded onto the surface of a tubular biomaterial.

26. The composition according to any of claims 20-25 wherein said tubular biomaterial is tubular decellularized animal vascular tissue.

27. The composition according to any of claims 20-25 wherein said tubular biomaterial is a tube formed from collagen or a mixture of collagen and a polymeric material.

28. A method for revascularizing damaged blood vessels and other vascular tissue comprising seeding the surface of damaged blood vessels and/or other vascular tissue with MesoT cells in combination with a differentiation medium for a period of at least 8-10 days to produce endothelial cells, smooth muscle cells and optionally pericytes in said vessels or tissue, which will self-assemble into vascular tissue, thus repairing the damaged blood vessels and/or vascular tissue.

29. The method according to claim 28 wherein said MesoT cells are seeded onto the surface of the vessels and/or tissue suspended in said media.

30. The method according to claim 28 or 29 wherein said media is chemically defined media (CDM) minus Activin A, further in combination with an effective amount of a VEGF agonist.

31. The method according to claim 30 wherein said VEGF agonist is a VEGF pathway agonist or a VEGF receptor agonist.

32. The method according to claim 30 or 31 wherein said VEGF agonist is VEGF-A₁₆₅.
